(19) United States
(12) Patent Application Publication
(10) Pub. No.: US 2005/0227230 A1
(43) Pub. Date: Oct. 13, 2005

(54) Detection of dna methylation

(75) Inventors: Bernard John Carroll, West End, Queensland (AU); Dion Kevin Harrison, Karana Downs (AU); Hnin Thanda Aung, Taringa (AU)

Correspondence Address: MORRISON & FOERSTER LLP, 1650 TYSONS BOULEVARD, SUITE 300, MCLEAN, VA, 22102, US

(73) Assignee: Davies Collison Cave, Milton, Queensland (AU)

(21) Appl. No.: 10/489,654

(22) PCT Filed: Sep. 13, 2002
(86) PCT No.: PCT/AU02/01262

§ 371 Date: Oct. 22, 2004

(30) Foreign Application Priority Data
Sep. 14, 2001 (AU) PR 7685

(51) Int. Cl. C12Q 1/68; C12P 19/34

(52) U.S. Cl. 435/6; 435/91.2

(57) ABSTRACT

The present invention relates generally to a method of genetic fingerprinting and more particularly to a method of establishing a methylation signature for a particular eukaryotic cell or group of cells such as in the form of tissue cells or organ-specific cells. The method of the present invention exploits the sensitivity of certain restriction enzymes to methylation. Exposure of genomic or transgene DNA to these enzymes followed by amplification results in products which establish the methylation signature or profile (i.e. the methylome profile) of a cell's or group of cells' genome when compared to the amplified genome in undigested form. The method of the present invention is useful inter alia in phenotyping a cell based on its methylation signature and provides a useful tool in functional genomics and for the design of therapeutic and trait-modifying protocols, particularly for animals and plants. The present invention can also be used to identify and map junctions between methylated and unmethylated DNA. The method of the present invention is also useful for identifying DNA methylation polymorphisms which can be used inter alia in diagnosis and forensics and for identifying particular genes, the function or absence of function of which are associated with a disease condition or trait. The method is also useful for monitoring the aging process of particular cells or an animal (including a human) or plant comprising such cells as well as monitoring the pluripotent or multipotent state of stem cells and development of stem cells through to maturation.

### FIELD OF THE INVENTION

The present invention relates generally to a method of genetic fingerprinting and more particularly to a method of establishing a methylation signature for a particular eukaryotic cell or group of cells such as in the form of tissue cells or organ-specific cells. The method of the present invention exploits the sensitivity of certain restriction enzymes to methylation. Exposure of genomic or transgene DNA to these enzymes followed by amplification results in products which establish the methylation signature or profile (i.e. the methylome profile) of a cell's or group of cells' genome when compared to the amplified genome in undigested form. The method of the present invention is useful inter alia in phenotyping a cell based on its methylation signature and provides a useful tool in functional genomics and for the design of therapeutic and trait-modifying protocols, particularly for animals and plants. The present invention can also be used to identify and map junctions between methylated and unmethylated DNA. The method of the present invention is also useful for identifying DNA methylation polymorphisms which can be used inter alia in diagnosis and forensics and for identifying particular genes, the function or absence of function of which are associated with a disease condition or trait. The method is also useful for monitoring the aging process of particular cells or an animal (including a human) or plant comprising such cells as well as monitoring the pluripotent or multipotent state of stem cells and development of stem cells through to maturation.

### BACKGROUND OF THE INVENTION

Bibliographic details of references provided in the subject specification are listed at the end of the specification.

Reference to any prior art in this specification is not, and should not be taken as, an acknowledgment or any form of suggestion that this prior art forms part of the common general knowledge in any country.

The Human Genome Sequencing Project has provided an invaluable and near-comprehensive database of human genes. The challenge now is to understand how the genome is expressed in a certain fashion and in particular cell types to produce the traits that ultimately determine the nature and quality of life. In recent years, it has become increasingly apparent that DNA methylation plays a key role in the regulation of gene expression in higher organisms. DNA methylation (mainly methylation of cytosines in CpG sites) is thought to direct chromatin compaction and generally the switching off of genes. The importance of DNA methylation has been highlighted by its involvement in several human diseases, including cancer, and the list of such diseases is expected to expand rapidly in the post genomics era.

Defining the genomic methylation signatures (or “methylomes”) of healthy and diseased cells could lead to the understanding, early diagnosis and treatment of many human diseases.

The evolutionary roles of DNA methylation in eukaryotes have been suggested to include host defence against transposons and retrotransposons (O'Neill et al., Nature 393: 68-72, 1998) and developmental and tissue-specific gene regulation (Walsh and Bestor, Genes Dev. 13: 26-34, 1999). The role of DNA methylation in developmental gene control has been the subject of considerable controversy (Yoder et al., Trends Genet. 13: 335-340, 1997). A role for DNA methylation in human disease has also been widely suggested but difficult to demonstrate unequivocally. Nevertheless, there is now compelling recent evidence of its involvement in several human genetic diseases, including Rett Syndrome, ICF and Fragile X (Heinrich, Current Biology 10: 60-63, 2000). These diseases have been found to be linked to mutations at cytosine nucleotides, or to mutations in the genes encoding proteins that are involved in DNA methylation (Hendrich, 2000, supra). DNA methylation has also been implicated in carcinogenesis. Large scale genome-wide hypomethylation was detected in many tumor cells compared with their normal control cells (Laird and Jaenisch, Annu. Rev. Genet. 30: 441-464,1996). This deficiency in methylation is thought to cause genetic instability and subsequent tumor progression (Lengauer et al., Proc. Natl. Acad. Sci. USA 94: 2545-2550, 1997). Conversely, in a number of cases, silenced tumor suppressor genes have been correlated with methylation of the promoter (Baylin and Herman, Trends Genet. 16: 168-174, 2000; Herman, Semin. Cancer Biol. 9: 359-367, 1999).

One of the problems limiting this important field of research has been the lack of efficient procedures for large scale assessment of DNA methylation at CpG sites around the genome.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc.

In accordance with the present invention, an amplification-based assay procedure has been developed to determine the methylation profile of nucleotides in the genome of a cell or group of cells. More particularly, the nucleotides are in the form of CpG or CpNpG sites. The ability to determine genomic and transgene methylomes in a cell or group of cells is an important tool in functional genomics and in developing the next generation of gene-expression modulating agents. Combining methylation profile with mapping enables a determination of the epigenetic consequences of internal and external stimuli. For example, methylation profiles may correlate with disease conditions or a propensity for a disease condition to develop or monitoring the aging process or the development process of cells. Furthermore, the methylation profile can be used to determine genes which either are expressed or are not expressed in certain disease states or with certain phenotypic traits. The identification of a condition or predisposition for development of a condition leads to the selection of therapeutic agents or protocols for the treatment of same.

The present invention is predicated in part on an amplification-based technology referred to herein as amplified methylation polymorphisms (AMP). The AMP technology determines the methylation profile of many thousands of CpG or CpNpG sites around the genome and provides a genetic profile of the methylation status of these sites. This genetic signature is the methylome fingerprint of a cell's or group of cells' genome. The present invention extends, however, to determination of methylation of sites other than CpG or CpNpG sites.

The AMP technology involves amplification of DNA markers in the form of small inverted repeats comprising the CpG or CpNpG sites but where amplification depends on the methylation status of the cytosines within the amplicon or nearby.

In one particularly useful embodiment, the protocol uses a single arbitrary decamer oligonucleotide primer containing the recognition sequences of a methylation-sensitive restriction enzyme. These short oligonucleotide primers containing such recognition sequences are referred to herein as AMP primers. The recognition sequences for the methylation-sensitive restriction enzyme are located in the middle of the primer followed by up to four selective nucleotides, extending to the 3′ end. AMP profiles are generated from both undigested genomic DNA and genomic DNA digested with the methylation sensitive enzyme. Comparison of the profiles from digested and undigested genomic DNA reveals three classes of AMP markers: digestion resistant (Class I) indicative of methylation, digestion sensitive (Class II) indicative of non-methylation, and digestion dependent (Class III). The nature of the last class of AMP markers is proposed to represent physically-linked cis-acting inhibitory sequences which suppress amplification of Class III markers from undigested template. Digestion with the enzyme removes the inhibitor from the amplicon, thereby allowing amplification. The digestion-dependent (Class III) markers are proposed to encompass a methylated restriction site or sites in the amplicon sequence flanked by a non-methylated restriction site and then the putative inhibitory sequence. Digestion-dependent markers represent, therefore, junctions between methylated and non-methylated DNA in the genome. Cloning, sequencing and mapping AMP markers shows that they often correspond to CpG islands, features known to be landmarks for genes in genomes. Southern analysis has confirmed the utility of AMP for detecting DNA methylation polymorphisms between genomes and that Class III AMPs represent the junction between methylated and unmethylated DNA in genomes.

The methylome profile of a cell's genome, therefore, is provided by the determination of AMP markers. It is proposed that changes in methylation profile indicates, determines or causes changes in patterns of gene expression and important biological phenotypes such as cancer and aging.

The terms “methylation signature”, “methylation profile” and “methylome” are used interchangeably throughout the specification and all indicate the methylation status of a cell's or group of cells' genome. They can also be used to determine the expression or absence of expression of particular genes. The method of the present invention is useful in determining methylation polymorphisms which can then be linked to particular traits and/or phenotypic characteristics or stages of aging or development and can also be useful in forensics to assist in better profiling a victim or perpetrator.

The present invention contemplates, therefore, a method for determining the methylation profile of one or more nucleotides at one or more sites within the genome of a eukaryotic cell or group of cells by obtaining a sample of genomic DNA from the cell or group of cells, digesting a sub-sample of the sample of genomic DNA with a methylation-sensitive enzyme having a recognition nucleotide sequence comprising a nucleotide capable of methylation at one or more of the above-mentioned sites, subjecting the digested DNA to an amplification reaction using primers comprising a nucleotide sequence capable of annealing to a non-cleaved form of a cleavable nucleotide sequence of the methylation-sensitive enzyme recognition sequence and subjecting the products of the amplification reaction to separation or other detection means relative to a control. The control comprises another sub-sample of the sample of genomic DNA not subjected to digestion by the methylation-sensitive enzyme but subjected to an amplification reaction using the same primers as for the digested DNA sample and then subjecting the products to the amplification reaction to the separation or other detection means wherein the presence of amplification products in enzyme digested and non-digested samples is indicative of a methylation-sensitive enzyme-digestion-resistant marker, the absence and presence of amplification products in enzyme digested and undigested samples, respectively, is indicative of a methylation-sensitive enzyme-digestion-sensitive marker and the presence and absence of amplification products in enzyme digested and undigested samples, respectively, is indicative of a methylation-sensitive enzyme-digestion-dependent marker.

The method of the present invention determines, therefore, a methylation profile comprising methylated or unmethylated CpG or CpNpGp islands characterized as being a methylation-sensitive enzyme-digestion-resistant marker, a methylation-sensitive enzyme digestion-sensitive marker or a methylation-sensitive enzyme-digestion-dependent marker. A methylation profile is developed showing the presence or absence of particular methylated CpG or CpNpGp islands as well as junction sites between methylated and unmethylated regions. These are the methylation-sensitive enzyme-digestion-dependent AMP markers.

Methylation enzymes contemplated herein include AatII, AciI, AclI, AgeI, AscI, AvaI, BamHI, BsaA1, BsaH1, BsiE, BsiW, BsrF, BssHII, BstBI, BstUI, Cla1, EagI, HaeII, HgaI, HhaI, HinPI, HpaII, MloI, MspI, NaeI, NarI, NotI, NruI and PmlI. HpaII is particularly preferred in accordance with the present invention.

Accordingly, another aspect of the present invention provides a method for determining the methylation profile of one or more CpG or CpNpG nucleotides at one or more sites within the genome of a eukaryotic cell or group of cells by obtaining a sample of genomic DNA from the cell or group of cells, digesting a sub-sample of the sample of genomic DNA with HpaII which has a recognition nucleotide sequence corresponding to or within the sites, subjecting the digested DNA to an amplification means such as polymerase chain reaction (PCR) using primers comprising a nucleotide sequence capable of annealing to a non-cleaved form of a HpaII cleavable nucleotide sequence and subjecting the products of the PCR to separation or other detection means relative to a control. The control comprises in this embodiment another sub-sample of the sample of genomic DNA not subjected to digestion by HpaII but subjected to an amplification reaction using the same primers as for the digested DNA sample and then subjecting the products to the amplification reaction to the separation or detection means wherein the presence of PCR products in enzyme digested and non-digested samples is indicative of a HpaII-digestion-resistant marker (H^{I}), the absence and presence of PCR products in enzyme digested and undigested samples, respectively, is indicative of a HpaII-digestion-sensitive marker (H^{S}) and the presence and absence of PCR products in enzyme digested and undigested samples, respectively, is indicative of a HpaII-digestion-dependent marker (H^{d}).

Another aspect of the present invention contemplates an assay device in the form of a kit useful for determining the methylation profile of one or more nucleotides at one or more sites within the genome of a eukaryotic cell, the kit comprising in compartmental form multiple compartments each adapted to comprise one or more of buffers, diluents and enyzmes as single or multiple components which are optionally required to be admixed prior to use, the kit further comprising instructions for use, wherein the method is conducted by obtaining a sample of genomic DNA from said cell, digesting a sub-sample of said sample of genomic DNA with a methylation-sensitive enzyme having a recognition nucleotide sequence comprising a nucleotide capable of being methylated at one or more of the above-mentioned sites, subjecting the digested DNA to an amplification reaction using primers comprising a nucleotide sequence capable of annealing to a non-cleaved form of a cleavable nucleotide sequence of the methylation-sensitive enzyme recognition sequence and subjecting the products of the amplification reaction to separation or detection means relative to a control, said control comprising another sub-sample of said sample of genomic DNA not subjected to digestion by the methylation-sensitive enzyme but subjected to an amplification reaction using the same primers as for the digested DNA sample and then subjecting the products to the amplification reaction to the separation or other detection means wherein the presence of amplification products in enzyme digested and non-digested samples is indicative of a methylation-sensitive enzyme-digestion-resistant marker, and wherein the absence and presence of amplification products in enzyme digested and undigested samples, respectively, is indicative of a methylation-sensitive enzyme-digestion-sensitive marker and wherein the presence and absence of amplification products in enzyme digested and undigested samples, respectively, is indicative of a methylation-sensitive enzyme-digestion-dependent marker.

Preferably, as indicated above, the methylation sensitive enzyme is HpaII. Conveniently, the amplification reaction is a PCR

The present method can also be used to detect a change in a cell's or group of cells' developmental state or a cell's or group of cells' exposure to an internal or external stimulus by detecting a change in methylation profile over time. This may be particularly relevant and useful in monitoring stem cells including stem cells in developmental transition and including embryonic stem (ES) cells, embryonic germ (EG) cells and mature or committed cells. It is also particularly useful in monitoring an animal or plant cell in tissue culture and/or following recombinant procedures. The method is also useful in forensics to identify potential characteristics of an individual based on the methylation profile of DNA samples from a victim or perpetrator. The methylation profile further provides an indicator as to the likely sensitivity of a cell or group of cells to a virus or transposon.

The method of the present invention is also useful for monitoring the state and/or extent of methylation of cloned, non-human embryos such as livestock embryos. These embryos can be checked for unwanted methylation prior to transplantation as well as after transplantation.

Alternatively, the present invention provides a method for detecting a change in a cell's or group of cells' physiological status such as from a normal state to a disease state.

### BRIEF DESCRIPTION OF THE FIGURES

FIG. 1 is a diagrammatic representation showing the AMP protocol and the Class I, II and III markers using as an example, the methylation-sensitive endonuclease restriction enzyme, HpaII.

FIG. 2 is a diagrammatic representation showing a Class III marker detected by the AMP protocol in one cell type but not another. Cloning, sequencing and Southern analysis has shown that inhibitory sequences are often associated with or linked to classical CpG islands.

FIG. 3 is photographic representation showing different classes of AMP markers. Comparison of profiles amplified with an AMP primer from either HpaII-digested (H) or undigested (U) genomic DNA. The profiles in the figure were generated from replicate DNA extractions from blood of a single human being. The figure demonstrates the reproducibility of the AMP protocol and the three classes of AMP markers: digestion sensitive (left-side arrows), digestion dependent (right-side arrows) and digestion resistant (without arrows).

FIG. 4 is a photographic representation showing tissue-specific AMP profiles observed for human blood and spermatozoa. Profiles were amplified from HpaII-digested (H) and undigested (U) genomic DNA of six humans (B1, B2, B3, S1, S2, and S3). Six different individuals contributed the samples; B1, B2, and B3 were blood samples, whereas S1, S2 and S3 were spermatozoa samples. Tissue specific digestion-dependent (upper panel) and digestion-sensitive (lower panel) DNA methylation polymorphisms (see arrows) were readily observed. DNA was extracted and analyzed in duplicate from each tissue sample.

FIG. 5 is a diagrammatic representation showing map location of four human digestion-dependent Class III AMP markers and the flanking HpaII sites. Each of these digestion-dependent markers that was cloned and sequenced was 100% homologous to sequenced regions of the human genome. The positions of the Class III amplicons (HdM 1-4) are indicated by thick shaded boxes on the chromosomal segment. The locations of HpaII sites are represented by vertical lines and the distance to, and orientation of nearby genes are indicated. Cloning, sequencing and mapping of these and additional Class III AMP markers has confirmed that they often map to CpG islands.

FIG. 6 is a photographic representation showing distinct methylomes of tissues from testis, peritoneal fat pad and bone marrow of mice.

FIG. 7 is a photographic representation showing widespread DNA methylation polymorphisms in mouse macrophage cell lines.

FIG. 8 is representation showing that AMP is a powerful diagnostic tool for identifying stable cell lines, e.g. sub-cultures (1, 2 and 3 in replicate) of the human colon carcinoma cell line HCT116 are identical for DNA methylation and DNA sequence.

FIG. 9 is a representation showing that AMP detects DNA methylation in a p53 knock-out line compared to the parent cell line HCT116. Both cell lines are stable between sub-cultures (1, 2 and 3), but AMP detects widespread DNA methylation and DNA sequence changes between the two cell lines.

FIG. 10 is a photographic representation showing that Southern hybridization confirms tissue-specific AMPs between sperm (S) and blood (B).

### DETAILED DESCRIPTION OF THE PREFERRED EMBODIMENTS

The present invention provides an amplification-based assay to determine the methylome's profile in the genome of a eukaryotic cell or group of cells.

Determination of the methylation profile includes determining a methylation fingerprint or signature of global methylation within a cell's or group of cell's genome or at specific sites or regions. The terms “methylome” and “methylomes” refer to the generally multiple sites of methylation within a cell's or group of cells' genome. These sites are also generally distinctive. The methylome profile is proposed to change with developmental changes, internal and external stimuli (e.g. environmental factors, RNAi, epigenetic signals) and disease conditions (e.g. cancer). The methylome profile also directs changes in gene expression in a cell. This can lead to different developmental, physiological or metabolic states or conditions. It can also lead to the identification of one or more genes or other genetic sequences such as promoter sequences involved or associated with a disease condition or trait. This is important, for example, during proliferation, development and self-renewal of precursor cells such as embryonic stem (ES) cells, embryonic germ (EG) cells and post-natal (e.g. adult) or committed cells. It is also relevant in monitoring plant or animal cells in tissue culture and/or following recombinant DNA procedures as well as monitoring the extent of methylation of animal embryos such as in the livestock industry. The embryos may be checked at pre- and post-transplantation stages.

Accordingly, one aspect of the present invention contemplates a method for determining the methylation profile of one or more nucleotides at one or more sites within the genome of a eukaryotic cell or group of cells, said method comprising obtaining a sample of genomic DNA from said cell or group of cells, digesting a sub-sample of said sample of genomic DNA with a methylation-sensitive enzyme having a recognition nucleotide sequence comprising a nucleotide capable of methylation at one or more of the above-mentioned sites, subjecting said digested DNA to an amplification reaction using primers comprising a nucleotide sequence capable of annealing to a non-cleaved form of a cleavable nucleotide sequence of the methylation-sensitive enzyme recognition sequence and subjecting the products of the amplification reaction to separation or other detection means relative to a control but subjected to an amplification reaction using the same primers as for the digested DNA sample and then subjecting the products of the amplification reaction to the separation and/or detection means, said control comprising another sub-sample of said sample of genomic DNA not subjected to digestion by the methylation-sensitive enzyme wherein the presence of amplification products in enzyme digested and non-digested samples is indicative of a methylation-sensitive enzyme-digestion-resistant marker, the absence and presence of amplification products in enzyme digested and undigested samples, respectively, is indicative of a methylation-sensitive enzyme-digestion-sensitive marker and the presence and absence of amplification products in enzyme digested and undigested samples, respectively, is indicative of a methylation-sensitive enzyme-digestion-dependent marker.

The method of this aspect of the present invention involves obtaining a sample of DNA and digesting one sub-sample with a methylation-sensitive enzyme and keeping another sub-sample undigested. Both samples are then subjected to amplification with a primer having a complementary sequence to a recognition sequence for the methylation-sensitive enzyme. If methylation has occurred at a particular site, the complementary sequence of the primer is intact as the enzyme is unable to digest the nucleotide sequence at that site. An amplification product is, therefore, present in the digested and undigested samples. If methylation has not occurred, digestion with the methylation sensitive enzyme destroys the primer-binding site. Consequently, the undigested sample will have an amplification product but the digested sample will not. Finally, if the methylated site is flanked or proximal to an amplification inhibiting region, the digestion will remove that region to enable an amplification product but this will be absent in the undigested sample due to the inhibition. This is regarded as a junction between methylated and unmethylated DNA. Thus, the AMP protocol detects junctions between methylated and unmethylated DNA in the genome.

In a preferred aspect of the present invention, a global methylation signature is determined using methylation-sensitive restriction endonuclease sites within islands of repeating nucleotides. The most preferred islands are referred to as CpG or CpNpG islands where N is any nucleotide. The most preferred methylation-sensitive restriction endonuclease sites within or comprising CpG or CpNpG islands are HpaII sites. A HpaII site is

```
  ↓
CCGG.
      ↑
```

Accordingly, another aspect of the present invention contemplates a method for determining the methylation profile of one or more CpG or CpNpG nucleotides at one or more sites within the genome of a eukaryotic cell, said method comprising obtaining a sample of genomic DNA from said cell, digesting a sub-sample of said sample of genomic DNA with HpaII which has a recognition nucleotide sequence corresponding to or within said sites, subjecting said digested DNA to an amplification reaction using primers comprising a nucleotide sequence capable of annealing to a non-cleaved form of a HpaII cleavable nucleotide sequence and subjecting the products of the amplification reaction to separation or other detection means relative to a control but subjected to an amplification reaction using the same primers as for the digested DNA sample and then subjecting the products of the amplification reaction to the separation or other detection means, said control comprising another sub-sample of said sample of genomic DNA not subjected to digestion by HpaII, wherein the presence of amplification products in enzyme digested and non-digested samples is indicative of a HpaII-digestion-resistant marker (H^{I}), the absence and presence of amplification products in enzyme digested and undigested samples, respectively, is indicative of a HpaII-digestion-sensitive marker (H^{S}) and the presence and absence of amplification products in enzyme digested and undigested samples, respectively, is indicative of a HpaII-digestion-dependent marker (H^{d}).

Whilst HpaII, and its functional equivalents, are the most convenient methylation-sensitive enzyme for use in the present invention up to the present time, one skilled in the art would immediately recognize that other enzymes may be employed with recognition nucleotide sequences within, comprising or adjacent to a CpG or CpNpG nucleotide sequences. A recognition nucleotide sequence is the sequence of nucleotides which is essential for the cleavage by a restriction endonuclease. A recognition nucleotide sequence is a sequence which is cleaved by the enzyme. A list of other enzymes which may be used include AatII, AciI, AclI, AgeI, AscI, AvaI, BamHI, BsaA1, BsaH1, BsiE, BsiW, BsrF, BssHII, BstBI, BstUI, Cla1, EagI, HaeII, HgaI, HhaI, HinPI, MloI, MspI, NaeI, NarI, NotI, NruI and PmlI.

The term “digestion” means that the DNA and restriction enzyme are brought together in physical biochemical contact for a time and under conditions sufficient for the cleavable nucleotide sequences to undergo cleavage. Digestion is generally for a time to permit cleavage of substantially all cleavable sites specific for the particular enzyme.

The term “genomic DNA” includes all DNA in a cell, group of cells, or in an organelle of a cell and includes exogenous DNA such a transgenes introduced into a cell.

Any amplification methodology may be employed in accordance with the present invention. Amplification methodologies contemplated herein include the polymerase chain reaction (PCR) such as disclosed in U.S. Pat. Nos. 4,683,202 and 4,683,195; the ligase chain reaction (LCR) such as disclosed in European Patent Application No. EP-A-320 308 and gap filling LCR (GLCR) or variations thereof such as disclosed in International Patent Publication No. WO 90/01069, European Patent Application EP-A-439 182, British Patent No. GB 2,225,112A and International Patent Publication No. WO 93/00447. Other amplification techniques include Qβ replicase such as described in the literature; Stand Displacment Amplification (SDA) such as described in European Patent Application Nos. EP-A-497 272 and EP-A-500 224; Self-Sustained Sequence Replication (3SR) such as described in Fahy et al. (PCR Methods Appl. 1(1): 25-33, 1991) and Nucleic Acid Sequence-Based Amplification (NASBA) such as described in the literature.

Some amplification reactions, for example, PCR and LCR, involve cycles of alternately high and low set temperatures, a process known as “thermal cycling”. PCR or “polymerase chain reaction” is an amplification reaction in which a polymerase enzyme, usually thermostable, generates multiple copies of the original sequence by extension of a primer using the original nucleic acid as a template. PCR is described in more detail in U.S. Pat. Nos. 4,683,202 and 4,683,195. LCR or “ligase chain reaction” is a nucleic acid amplification reaction in which a ligase enzyme, usually thermostable, generates multiple copies of the original sequence by ligating two or more oligonucleotide probes while they are hybridized to the target. LCR and its variation, Gap LCR, are described in more detail in European Patent Application Nos. EP-A-320-308 and EP-A-439-182 and International Patent Publication No. WO 90/100447 and elsewhere.

The PCR amplification process is the most preferred in practising the present invention.

Accordingly, in a preferred aspect of the present invention, there is provided a method for determining the methylation profile of one or more CpG or CpNpG nucleotides at one or more sites within the genome of a eukaryotic cell or group of cells, said method comprising obtaining a sample of genomic DNA from said cell or group of cells, digesting a sub-sample of said sample of genomic DNA with HpaII which has a recognition nucleotide sequence corresponding to or within said sites, subjecting said digested DNA to a polymerase chain reaction (PCR) using primers comprising a nucleotide sequence capable of annealing to a non-cleaved form of a HpaII cleavable nucleotide sequence and subjecting the products of the PCR to separation or other detection means relative to a control but subjected to an amplification reaction using the same primers as for the digested DNA sample and then subjecting the products of the amplification reaction to the separation or other detection means, said control comprising another sub-sample of said sample of genomic DNA not subjected to digestion by HpaII wherein the presence of PCR products in enzyme digested and non-digested samples is indicative of a HpaII-digestion-resistant marker (H^{I}), the absence and presence of PCR products in enzyme digested and undigested samples, respectively, is indicative of a HpaII-digestion-sensitive marker (H^{S}) and the presence and absence of PCR products in enzyme digested and undigested samples, respectively, is indicative of a HpaII-digestion-dependent marker (H^{d}).

The term “separation means” extends to any profiling protocol aimed at distinguishing between different amplification products. The term “separation means” may also be read as “separation or other detection means”. Gel electrophoresis is the most convenient as this permits comparison of individual bands following separation. The term “gel” includes agarose and polyacrylamide amongst others. A range of other separation means may also be applied such as those involving spectrophotometric, fluorometric and mass spectrographic methods. These latter methods are particularly useful for detecting a specific amplicon or amplicons.

In one particular embodiment, the presence or absence of AMP markers is assayed in real-time using a real-time PCR. As indicated above, PCR is well known to those in the art. A real-time PCR reaction includes a PCR reaction in which the products are detected during the PCR reaction. Preferably, the detection of the PCR product or products is made during the linear phase of a PCR reaction. It is known to those skilled in the art that, during the linear phase of a PCR, the quantity of a product or amplicon formed is proportional to the quantity of target sequence present at the initiation of the PCR.

In a preferred real-time PCR, a labeled probe molecule capable of hybridizing to the target sequence (e.g. an AMP marker) is used in the PCR. In subsequent amplification rounds, the probe is displaced from the amplicon by the 5′ exonuclease activity of Taq polymerase. Generally, but not exclusively, the probes are labeled at the 5′ nucleotide and the exonuclease activity releases the 5′ probe molecule. Release of the labeled nucleotide from the probe results in the ability to detect the presence of a specific PCR product. Real-time PCR assays are well known to those skilled in the art and include those disclosed in U.S. Pat. No. 6,277,638.

The term “label” means a moiety which is conjugated to a probe or an oligonucleotide moiety. A labeled probe or oligonucleotide moeity can be detected, identified or captured by virtue of the label of moiety which is conjugated to the probe. A “directly detectable” label is a signal-producing label which is capable of detection either directly or through its interaction with a substance such as a substrate (in the case of an enzyme), a light source (in the case of a fluorescent compound) or a photomultiplier tube (in the case of a radioactive or chemiluminescent compound). A “proximity label” is one of at least two labels which interact with each other to produce a detectable signal when the proximity labels are brought together. Typically, a first proximity label is used in combination with a corresponding second proximity label. An “indirectly detectable” label is a substance which in and of itself does not provide a signal but which can be utilized to identify an oligonucleotide to which the indirectly detectable label is attached. For example, biotin can be an indirectly detectable label.

DNA molecules are said to have a “5′-terminus” (5′ end) and a “3′-terminus” (3′ end) because nucleic acid phosphodiester linkages occur to the 5′ carbon and 3′ carbon of the pentose ring of the substituent mononucleotides. A terminal nucleotide as used herein is the nucleotide at the end position of the 3′- or 5′-terminus. As used herein, a nucleotide sequence, even if internal to a larger oligonucleotide or polynucleotide, can also be said to have 5′- and 3′-ends.

Reference herein to a “multiplex PCR” refers to a real-time PCR that contains more than one probe. In this embodiment, AMP marker separation is achieved by monitoring either the accumulation of a specific labeled nucleotide or monitoring the depletion of a specifically labeled nucleotide from the PCR.

Accordingly, another aspect of the present invention contemplates a method for determining the methylation profile of one or more nucleotides at one or more sites within the genome of a eukaryotic cell or group of cells, said method comprising obtaining a sample of genomic DNA from said cell or group of cells, digesting a sub-sample of said sample of genomic DNA with a methylation-sensitive enzyme having a recognition nucleotide sequence comprising a nucleotide capable of methylation at one or more of the above-mentioned sites, subjecting said digested DNA to an amplification reaction using primers comprising a nucleotide sequence capable of annealing to a non-cleaved form of a cleavable nucleotide sequence of the methylation-sensitive enzyme recognition sequence and subjecting the products of the amplification reaction to real-time PCR to distinguish amplification products relative to a control but subjected to an amplification reaction using the same primers as for the digested DNA sample and then subjecting the products of the amplification reaction to the separation or other detection means, said control comprising another sub-sample of said sample of genomic DNA not subjected to digestion by the methylation-sensitive enzyme wherein the presence of amplification products in enzyme digested and non-digested samples is indicative of a methylation-sensitive enzyme-digestion-resistant marker, the absence and presence of amplification products in enzyme digested and undigested samples, respectively, is indicative of a methylation-sensitive enzyme-digestion-sensitive marker and the presence and absence of amplification products in enzyme digested and undigested samples, respectively, is indicative of a methylation-sensitive enzyme-digestion-dependent marker.

More particularly, the present invention provides a method for determining the methylation profile of one or more CpG or CpNpG nucleotides at one or more sites within the genome of a eukaryotic cell or group of cells, said method comprising obtaining a sample of genomic DNA from said cell, digesting a sub-sample of said sample of genomic DNA with HpaII which has a recognition nucleotide sequence corresponding to or within said sites, subjecting said digested DNA to an amplification reaction using primers comprising a nucleotide sequence capable of annealing to a non-cleaved form of a HpaII cleavable nucleotide sequence and subjecting the products of the amplification reaction to distinguish amplification products relative to a control but subjected to an amplification reaction using the same primers as for the digested DNA sample and then subjecting the products of the amplification reaction to the separation or other detection means, said control comprising another sub-sample of said sample of genomic DNA not subjected to digestion by HpaII, wherein the presence of amplification products in enzyme digested and non-digested samples is indicative of a HpaII-digestion-resistant marker (H^{I}), the absence and presence of amplification products in enzyme digested and undigested samples, respectively, is indicative of a HpaII-digestion-sensitive marker (H^{S}) and the presence and absence of amplification products in enzyme digested and undigested samples, respectively, is indicative of a HpaII-digestion-dependent marker (H^{d}).

Reference herein to a “probe molecule” refers to a labeled nucleotide molecule which is capable of hybridizing to a target sequence. A target sequence is generally a DNA amplicon produced by a first amplification reaction following restriction digestion by a methylation-sensitive enzyme. Methods for the production of nucleic acid probes suitable for use in PCR are well known in the art. Methods for the determination of a suitable size and sequence of an oligonucleotide are also well known in the art.

In one particularly useful embodiment, the probe molecule is fluorescently labeled at its 5′ end.

Fluorescent nucleotide analogs are widely known and commercially available from several sources. An exemplary source is NEN (trademark) Life Science Products (Boston, Mass., USA) which offers dideoxy-, deoxy- and ribonucloetide analogs labeled with fluorescein, courmarin, tetramethylrhodamine, naphthofluorescein, pyrene, Texas Red (registered trademark) and Lissamine (trademark). Other suppliers include Amersham Pharmacia Biotech (Uppsala, Sweden; Piscataway, N.J., USA) and MBI Fermentas, Inc. (Amherst, N.Y., USA).

An advantage to using fluorescent labels and fluorescence spectroscopic analysis is that there are multiple different labels available. Such different labels can be particularly useful in a multiplex embodiment of the present invention. Different fluorescent labels may be used in different probes so that the detection of a particular fluorescently-labeled nucleotide can be distinguished from others and used to deduce which AMP markers are present.

For example, fluorescein has a 488 nm excitation and 520 nm emission wavelength, whereas rhodamine (in the form of tetramethylrhodamine) has 550 nm excitation and 575 nm emission wavelength. A fluorescence detector provides an excitation source and an emission detector. The emission wavelengths of 520 nm and 575 nm are easily distinguishable using fluorescence spectroscopy.

On a per molecule basis, fluorescence spectroscopy is about 10-fold more sensitive than absorbance spectroscopy. A very wide variety of fluorescence spectroscopy-based detectors are commercially available for reading fluorescence values of single tubes, flow cells and multi-well plates, amongst others. For example, Labsystems Multiskan models of microplate readers are widely available with a spectral range of 400-750 nm and filters for 340, 405, 414, 450, 492, 540, 620 and 690 nm (e.g. Fisher Scientific, Pittsburgh, Pa., USA).

In a related embodiment, the present invention contemplates the use of fluorescent resonant energy transfer (FRET) for the detection of a PCR amplicon. FRET is a form of molecular energy transfer by which energy is passed between a donor molecule and an acceptor molecule. FRET arises from the properties of certain chemical compounds. When excited by exposure to particular wavelengths of light, they emit light (i.e. they fluoresce) at a different wavelength. Such compounds are termed “fluorophores”. In FRET, energy is passed over a long distance between a donor molecule which is a fluorophore and an acceptor molecule. The donor absorbs a photon and transfers energy to the acceptor.

Molecules that are commonly used in FRET include fluorescein, 5-carboxyfluorescein (FAM), 2′7′ dimethoxy-4′5′-dichloro-6-carbosyfjuorescein (JOI), rhodamine, 6-carboxy-rhodamine (R6G), N,N,N,N-tetramethyl-6-carboxyrhodamine (TAMRA), 6-carboxy-X-rhodamine (ROX), 4-(4′-dimethylaminophenylazo) benzoic acid (DABCYL) and 5-(2′-aminoethyl)aminonaphthalene-1-sulfonic acid (EDANS).

In another aspect of the present invention, absorbance spectrographic analysis is contemplated to provide an analysis of AMP markers generated in a PCR reaction thereby providing for the determination of the methylation status of a gene. In this aspect of the present invention, a chromatographic separation with light absorbance detection of an AMP marker amplicon in a PCR mixture can be used to measure the quantity of PCR product. In this case, the second PCR employs a specific probe/primer to identify a particular amplicon. In an illustrative embodiment, a multiplexed assay for the presence of several different nucleic acid target sequences in a sample is analyzed by absorbance spectroscopy. Several labeled probes to various nucleic acid target sequences are added to a nucleic acid sample. The labels on the probes may be various nucleotide analogs, a different one for each probe. A PCR reaction is conducted and the quantity of a PCR product is measured by adsorbance spectroscopy. In this aspect of the present invention, the PCR is directly loaded onto a pre-equilibrated high pressure liquid chromatography (HPLC) column and eluted under conditions that separate the nucleotide analogs from the natural nucleotides. Useful media for chromatographic separation of nucleotides, bases and nucleosides include reverse phase media (such as a reverse phase C18 column or ODS-80Tₘ or ODS-120T TSK-GEL by TosoHaas [Montgomeryville, Pa., USA]), anion exchange media (such as DEAE-25SW or SP-25W TSK-GEL by TosoHaas [Montgomeryville, Pa., USA]) or affinity media (such as Boronate-5PW TSK-GEL by [Montgomeryville, Pa., USA]).

The HPLC column is fitted with an absorbance detector to monitor the column effluent, hence, “absorbance spectroscopy” for this type of analysis. Typical wavelengths for monitoring HPLC detection of nucleotides are 250 nm, 260 nm and 280 nm. Thus, the quantity of a PCR product may be determined in real-time by periodic sampling of the PCR.

Identification of the AMP marker PCR products can be accomplished by comparison of the retention times (as monitored by absorbance of effluent at various times) of standards of the nucleotide analogs separated on the same HPLC column under the same conditions. Alternatively, the identity of the nucleotide analogs collected in separate fractions after chromatographic separation (as determined by monitoring the absorbance of the column effluent) can be determined by other standard analytical methods such as gel electrophoresis. In an alternative aspect, the PCR mixture is separated on a gas chromatograph fitted with an absorbance detector to monitor column effluent.

In another embodiment, the AMP marker or multiplex of AMP markers generated in a single PCR reaction are hybridized to a microarray of potentially complementary nucleotide sequences such as DNA sequences. As a single oligonucleotide of the present invention (comprising, for example, a HpaII site) may generate a plurality of AMP markers belonging to different AMP classes, a rapid method is required to determine the nucleotide sequence on the AMP markers. The nucleotide sequence identity of a particular AMP marker can be determined by reference, for example, to a known genomic DNA sequence. Suitable arrays of DNA for the analysis of AMP markers include microarrays of human genes and/or contigs derived from genomic libraries, or particularly, arrays or CpG islands in genomes. Hybridization of a PCR product comprising one or a plurality of AMP markers facilitates the isolation and identification of genetic sequences.

A “nucleic acid” as used herein, is a covalently linked sequence of nucleotides in which the 3′ position of the phosphorylated pentose of one nucleotide is joined by a phosphodiester group to the 5′ position of the pentose of the next nucleotide and in which the nucleotide residues are linked in specific sequence; i.e. a linear order of nucleotides. A “polynucleotide” as used herein, is a nucleic acid containing a sequence that is greater than about 100 nucleotides in length. An “oligonucleotide” as used herein, is a short polynucleotide or a portion of a polynucleotide. An oligonucleotide typically contains a sequence of about two to about one hundred bases. The word “oligo” is sometimes used in place of the word “oligonucleotide”. The term “oligo” also includes a particularly preferred primer length in the practice of the present invention of up to about 10 nucleotides.

As used herein, the term “primer” refers to an oligonucleotide or polynucleotide that is capable of hybridizing to another nucleic acid of interest under particular stringency conditions. A primer may occur naturally as in a purified restriction digest or be produced synthetically, by recombinant means or by PCR amplification. The terms “probe” and “primers” may be used interchangeably, although to the extent that an oligonucleotide is used in a PCR or other amplification reaction, the term is generally “primer”. The ability to hybridize is dependent in part on the degree of complementarity between the nucleotide sequence of the primer and complementary sequence on the target DNA.

The terms “complementary” or “complementarity” are used in reference to nucleic acids (i.e. a sequence of nucleotides) related by the well-known base-pairing rules that A pairs with T or U and C pairs with G. For example, the sequence 5′-A-G-T-3′ is complementary to the sequence 3′-T-C-A-5′ in DNA and 3′-U-C-A-5′ in RNA. Complementarity can be “partial” in which only some of the nucleotide bases are matched according to the base pairing rules. On the other hand, there may be “complete” or “total” complementarity between the nucleic acid strands when all of the bases are matched according to base-pairing rules. The degree of complementarity between nucleic acid strands has significant effects on the efficiency and strength of hybridization between nucleic acid strands as known well in the art. This is of particular importance in detection methods that depend upon binding between nucleic acids, such as those of the invention. The term “substantially complementary” is used to describe any primer that can hybridize to either or both strands of the target nucleic acid sequence under conditions of low stringency as described below or, preferably, in polymerase reaction buffer (Promega, M195A) heated to 95° C. and then cooled to room temperature. As used herein, when the primer is referred to as partially or totally complementary to the target nucleic acid, that refers to the 3′-terminal region of the probe (i.e. within about 10 nucleotides of the 3′-terminal nucleotide position).

Reference herein to a stringency in relation to hybridization includes and encompasses from at least about 0 to at least about 15% v/v formarnide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur and Doty, J. Mol. Biol. 5: 109,1962). However, the Tₘ of a duplex DNA decreases by 1° C. with every increase of 1% in the number of mismatch base pairs (Bonner and Laskey, Eur. J. Biochem. 46: 83, 1974). Formamide is optional in these hybridization conditions. Accordingly, particularly preferred levels of stringency are defined as follows: low stringency is 6×SSC buffer, 0.1% w/v SDS at 25-42C; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The present invention provides, therefore, a methylation profile of the genome of a eukaryotic cell or group of cells, the methylation profile comprising the presence or absence of methylation at particular sites and/or junctions between methylated and unmethylated regions, said method comprising obtaining a sample of genomic DNA from said cell or group of cells, digesting a sub-sample of said sample of genomic DNA with a methylation-sensitive enzyme having a recognition nucleotide sequence comprising a nucleotide capable of methylation at one or more of the above-mentioned sites, subjecting said digested DNA to an amplification reaction using primers comprising a nucleotide sequence capable of annealing to a non-cleaved form of a cleavable nucleotide sequence of the methylation-sensitive enzyme recognition sequence and subjecting the products of the amplification reaction to separation or other detection means relative to a control but subjected to an amplification reaction using the same primers as for the digested DNA sample and then subjecting the products of the amplification reaction to the separation or other detection means, said control comprising another sub-sample of said sample of genomic DNA not subjected to digestion by the methylation-sensitive enzyme wherein the presence of amplification products in enzyme digested and non-digested samples is indicative of a methylation-sensitive enzyme-digestion-resistant marker, the absence and presence of amplification products in enzyme digested and undigested samples, respectively, is indicative of a methylation-sensitive enzyme-digestion-sensitive marker and the presence and absence of amplification products in enzyme digested and undigested samples, respectively, is indicative of a methylation-sensitive enzyme-digestion-dependent marker.

The present invention also contemplates kits for determining the methylation profile of one or more nucleotides at one or more sites within the genome of a eukaryotic cell or group of cells. The kits may comprise many different forms but in a preferred embodiment, the kits are designed to create products capable of resolution via electrophoresis.

The kit may also comprise instructions for use.

Accordingly, another aspect of the present invention contemplates an assay device in the form of a kit useful for determining the methylation profile of one or more nucleotides at one or more sites within the genome of a eukaryotic cell or group of cells, said kit comprising in compartmental form multiple compartments each adapted to comprise one or more of buffers, diluents and enyzmes as single or multiple components which are optionally required to be admixed prior to use, said kit further comprising instructions for use, wherein the method is conducted by obtaining a sample of genomic DNA from said cell or group of cells, digesting a sub-sample of said sample of genomic DNA with a methylation-sensitive enzyme having a recognition nucleotide sequence comprising a nucleotide capable of being methylated at one or more of the above-mentioned sites, subjecting said digested DNA to an amplification reaction using primers comprising a nucleotide sequence capable of annealing to a non-cleaved form of a cleavable nucleotide sequence of the methylation-sensitive enzyme recognition sequence and subjecting the products of the amplification reaction to separation or other detection means relative to a control but subjected to an amplification reaction using the same primers as for the digested DNA sample and then subjecting the products of the amplification reaction to the separation or other detection means, said control comprising another sub-sample of said sample of genomic DNA not subjected to digestion by the methylation-sensitive enzyme, wherein the presence of amplification products in enzyme digested and non-digested samples is indicative of a methylation-sensitive enzyme-digestion-resistant marker, and wherein the absence and presence of amplification products in enzyme digested and undigested samples, respectively, is indicative of a methylation-sensitive enzyme-digestion-sensitive marker and wherein the presence and absence of amplification products in enzyme digested and undigested samples, respectively, is indicative of a methylation-sensitive enzyme-digestion-dependent marker.

More particularly, another aspect of the present invention contemplates an assay device in the form of a kit useful for determining the methylation profile of one or more nucleotides at one or more sites within the genome of a eukaryotic cell or group of cells, said kit comprising in compartmental form multiple compartments each adapted to comprise one or more of buffers, diluents and enyzmes as single or multiple components which are optionally required to be admixed prior to use, said kit further comprising instructions for use, wherein the method is conducted by obtaining a sample of genomic DNA from said cell or group of cells, digesting a sub-sample of said sample of genomic DNA with HpaII which has a recognition nucleotide sequence corresponding to or within said sites, subjecting said digested DNA to an amplification reaction using primers comprising a nucleotide sequence capable of annealing to a non-cleaved form of a HpaII cleavable nucleotide sequence and subjecting the products of the amplification reaction to separation or other detection means relative to a control but subjected to an amplification reaction using the same primers as for the digested DNA sample and then subjecting the products of the amplification reaction to the separation or other detection means, said control comprising another sub-sample of said sample of genomic DNA not subjected to digestion by HpaII, wherein the presence of amplification products in enzyme digested and non-digested samples is indicative of a HpaII-digestion-resistant marker (H^{I}), the absence and presence of amplification products in enzyme digested and undigested samples, respectively, is indicative of a HpaII-digestion-sensitive marker (H^{S}) and the presence and absence of amplification products in enzyme digested and undigested samples, respectively, is indicative of a HpaII-digestion dependent marker (H^{d}).

Conveniently, the kits are adapted to contain compartments for two or more of the above-listed components. Furthermore, buffers, nucleotides and/or enzymes may be combined into a single compartment.

The Taq polymerase is an example of a suitable DNA polymerase which is thermostable and which is used in PCR and other amplification reactions. The thermostable DNA polymerase is used in an amount sufficient for a hybridized probe to release an identifier nucleotide. This amount may vary with the enzyme used and also with the temperature at which depolymerization is carried out. An enzyme of a kit is typically present in an amount sufficient to permit the use of about 0.1 to 100 U/reaction; in particularly preferred embodiments, the concentration is about 0.5 U/reaction. The Stoffel fragment is another example of a suitable DNA polymerase and is in fact preferred over Taq for AMPs.

As stated above, instructions optionally present in such kits instruct the user on how to use the components of the kit to perform the various methods of the present invention. It is contemplated that these instructions include a description of the detection methods of the subject invention, including detection by gel electrophoresis.

The present invention further contemplates kits which contain a primer for a nucleic acid target of interest with the primer being complementary to a predetermined nucleic acid target, such as comprising the complementary sequence or sense sequence of a HpaII recognition sequence. In another embodiment, the kit contains multiple primers or probes, each of which contains a different base at an interrogation position or which is designed to interrogate different target DNA sequences. In a contemplated embodiment, multiple probes are provided for a set of nucleic acid target sequences that give rise to analytical results which are distinguishable for the various probes.

It is contemplated that a kit comprises a vessel containing a purified and isolated enzyme whose activity is to release one or more nucleotides from the 3′ terminus of a hybridized nucleic acid probe and a vessel containing pyrophosphate. In one embodiment, these items are combined in a single vessel. It is contemplated that the enzyme is either in solution or provided as a solid (e.g. as a lyophilized powder); the same is true for the pyrophosphate. Preferably, the enzyme is provided in solution. Some contemplated kits contain labeled nucleic acid probes. Other contemplated kits further comprise vessels containing labels and vessels containing reagents for attaching the labels. Microtiter trays are particularly useful and these may comprise from two to 100,000 wells or from about six to about 10,000 wells or from about six to about 1,000 wells.

The methylation profile of a cell's or group of cells' genome may be in any location including promoter regions, 5′ regions prior to the authentic transcription start site, within coding (i.e. exon) and non-coding (i.e. intron) regions of genes and in the 3′ regions following the authentic termination signal. The methylome may also be in regulatory genes, structural genes and in non-protein-encoding genes.

The present invention has a range of utilities. For example, a number of cancers have been detected in which previously silenced genes (such as silenced at birth) are now expressed. A change in methylation profile, especially if mapped to cancer-associated genes, provides a diagnostic application for the AMP technology. Indeed, many complex medical disorders are likely to have a methylation component. For example, during hypertrophy in heart disease, there is a reappearance of expression of juvenile-expressed genes. This is presumably due to changes in methylation profile. In addition, methylation profiles may be implicated in the aging process as well as in cell development such as the development of stem cells (e.g. ES cells, post-natal stem cells) or EG cells to mature cells or cells of a developmental state in between ES cells and mature cells. Still further, the extent or type of methylation may be predictive of the sensitivity or otherwise of a cell's or group of cells' sensitivity to viruses or to transposon mutagenesis or transgene expression.

In a related embodiment, changes in methylation profile may, in one embodiment, be associated with RNAi. It is proposed that RNAi is induced by double-stranded RNA whether naturally occurring or artificially produced via co-suppression constructs. A change in methylome profile may be a result of a change in RNAi-specific modulation of gene expression. The detection of a change in methylome profile may provide a target for gene therapy. Furthermore, a change in methylation profile may be directly or indirectly induced.

As indicated above, methylation may also be a key element in the aging process. Modification of methylation profiles may delay the onset of or ameliorate the effects of age-related conditions and illnesses. Support for this proposition is in the change in methylation profiles in identical twins. The detection of age-related methylation profiles or polymorphisms is useful for example, in forensic medicine and testing of age of animals in the livestock industry.

The methylation profiling assay of the present invention is particularly useful, therefore, in monitoring aging of cells and may be used in conjunction with pallative care or in clinical management of individuals having the methylation profile of a cell or group of cells at a particular stage of development. This is important not only in human and veterinary health but also in monitoring plants and trees, especially those generated through tissue culture.

Another important application is in the high throughput screening of agents which are capable of demethylating genomes. This may be important, for example, in de-differentiating cells.

In relation to these embodiments, genetic constructs such as used, for example, in gene therapy, can be engineered to attach cell-specific methylation modulation signals to ensure that genes are regulated in the appropriate manner.

In the livestock industry, such as meat production, wool production and breeding programs, amongst many other facets, monitoring methylome profiles in relation to environmental conditions (e.g. drought, weather, change in diet, etc.) or following vaccination programs will be important for ensuring there is no loss of quality or decrease in production levels. This is also important for general toxicity testing of humans and animals. Many disease conditions are mediated by toxins and environmental contaminants and AMP technology provides a means for monitoring for changes in methylome profile.

Accordingly, another aspect of the present invention contemplates a method for detecting a change in a cell's or group of cells' developmental state or a cell's or group of cells' exposure to an internal or external stimulus, said method comprising detecting a change in methylation profile over time by the method comprising obtaining a sample of genomic DNA from said cell or group of cells, digesting a sub-sample of said sample of genomic DNA with a methylation-sensitive enzyme having a recognition nucleotide sequence comprising a nucleotide capable of being methylated at one or more of the above-mentioned sites, subjecting said digested DNA to an amplification reaction using primers comprising a nucleotide sequence capable of annealing to a non-cleaved form of a cleavable nucleotide sequence of the methylation-sensitive enzyme recognition sequence and subjecting the products of the amplification reaction to separation or other detection means relative to a control but subjected to an amplification reaction using the same primers as for the digested DNA sample and then subjecting the products of the amplification reaction to the separation or other detection means, said control comprising another sub-sample of said sample of genomic DNA not subjected to digestion by the methylation-sensitive enzyme, wherein the presence of amplification products in enzyme digested and non-digested samples is indicative of a methylation-sensitive enzyme-digestion-resistant marker, and wherein the absence and presence of amplification products in enzyme digested and undigested samples, respectively, is indicative of a methylation-sensitive enzyme-digestion-sensitive marker and wherein the presence and absence of amplification products in enzyme digested and undigested samples, respectively, is indicative of a methylation-sensitive enzyme-digestion-dependent marker.

More particularly, the present invention provides a method for detecting a change in a cell's or group of cells' developmental state or a cell's or group of cells' exposure to an internal or external stimulus, said method comprising detecting a change in methylation profile over time by the method comprising obtaining a sample of genomic DNA from said cell or group of cells, digesting a sub-sample of said sample of genomic DNA with HpaII which has a recognition nucleotide sequence corresponding to or within said sites, subjecting said digested DNA to an amplification reaction using primers comprising a nucleotide sequence capable of annealing to a non-cleaved form of a HpaII cleavable nucleotide sequence and subjecting the products of the amplification reaction to separation and/or detection means relative to a control but subjected to an amplification reaction using the same primers as for the digested DNA sample and then subjecting the products of the amplification reaction to the separation or other detection means, said control comprising another sub-sample of said sample of genomic DNA not subjected to digestion by HpaII wherein the presence of amplification products in enzyme digested and non-digested samples is indicative of a HpaII-digestion-resistant marker (H^{I}), the absence and presence of amplification products in enzyme digested and undigested samples, respectively, is indicative of a HpaII-digestion-sensitive marker (H^{S}) and the presence and absence of amplification products in enzyme digested and undigested samples, respectively, is indicative of a HpaII-digestion-dependent marker (H^{d}).

In a further embodiment, the present invention provides a method for detecting a change in a cell's or group of cells' physiological status such as from a normal state to a disease state, said method comprising obtaining a sample of genomic DNA from said cell or group of cells, digesting a sub-sample of said sample of genomic DNA with a methylation-sensitive enzyme having a recognition nucleotide sequence comprising a nucleotide capable of being methylated at one or more of the above-mentioned sites, subjecting said digested DNA to an amplification reaction using primers comprising a nucleotide sequence capable of annealing to a non-cleaved form of a cleavable nucleotide sequence of the methylation-sensitive enzyme recognition sequence and subjecting the products of the amplification reaction to separation or other detection means relative to a control but subjected to an amplification reaction using the same primers as for the digested DNA sample and then subjecting the products of the amplification reaction to the separation or other detection means, said control comprising another sub-sample of said sample of genomic DNA not subjected to digestion by the methylation-sensitive enzyme, wherein the presence of amplification products in enzyme digested and non-digested samples is indicative of a methylation-sensitive enzyme-digestion-resistant marker, and wherein the absence and presence of amplification products in enzyme digested and undigested samples, respectively, is indicative of a methylation-sensitive enzyme-digestion-sensitive marker and wherein the presence and absence of amplification products in enzyme digested and undigested samples, respectively, is indicative of a methylation-sensitive enzyme-digestion-dependent marker.

In yet another embodiment, the present invention provides a method for detecting a change in a cell's or group of cells' physiological status such as from a normal state to a disease state, said method comprising detecting a change in methylation profile over time by the method comprising obtaining a sample of genomic DNA from said cell or group of cells, digesting a sub-sample of said sample of genomic DNA with HpaII which has a recognition nucleotide sequence corresponding to or within said sites, subjecting said digested DNA to an amplification reaction using primers comprising a nucleotide sequence capable of annealing to a non-cleaved form of a HpaII cleavable nucleotide sequence and subjecting the products of the amplification reaction to separation or other detection means relative to a control but subjected to an amplification reaction using the same primers as for the digested DNA sample and then subjecting the products of the amplification reaction to the separation or other detection means, said control comprising another sub-sample of said sample of genomic DNA not subjected to digestion by HpaII wherein the presence of amplification products in enzyme digested and non-digested samples is indicative of a HpaII-digestion-resistant marker (H^{I}), the absence and presence of amplification products in enzyme digested and undigested samples, respectively, is indicative of a HpaII-digestion-sensitive marker (H^{S}) and the presence and absence of amplification products in enzyme digested and undigested samples, respectively, is indicative of a HpaII-digestion-dependent marker (H^{d}).

Preferably, the amplification reaction is PCR.

Disease conditions associated with methylation include but are not limited to Rett syndrome, ICF and Fragile X syndrome. Furthermore, the change in methylation profile can correlate with the change in expression patterns of particular genes and, hence, the assay of the present invention is a useful way of identifying new genes (or promoters or other genetic sequences) or for functionalizing a known gene or genetic sequence).

A change in methylome profile over time means that at least two time points are measured and there is a change in methylation profile over at least the two time points.

The present invention further permits the identification of genes and promoters having CpG or CpNpG islands which have HpaII sites or other methylation-sensitive restriction sites. Furthermore, application of the AMP technology permits identification of Class III markers (i.e. H^{d} sites). The identification of these sites permits identification of potential regulatory regions which can be targeted for agonists or antagonists of gene expression.

The present invention further contemplates mapping methylome profiles with phenotypic and/or genotypic profiles. A database of methylomes can then be produced relative to a phenotypic trait or a particular genotype. Such a database is useful in bioinformatics to establish likely phenotypes of, for example, animal, mammalian, avian, insect, reptillian, amphibian, fungal, yeast, nematode or other cells of a eukaryotic organism. Furthermore, the database may also be used to map junctions between methylated and non-methylated DNA and their association with important biological phenotypes. The latter frequently show high levels of cell specificity. The present invention further contemplates any method for monitoring these junctions and the association of these junctions with important biological phenotypes.

Methylation profiles are also important in plant tissue culture and/or in recombinant protocols applied to plants.

Thus, in another aspect, the invention contemplates a computer program product for assessing the likely phenotype of a cell based on methylome profile, said product comprising:

(1) code that receives an input value for one or more of features wherein said features are selected from:
(a) absence or presence of AMP marker Class I;
(b) absence or presence of AMP marker Class II:
(c) absence or presence of AMP marker Class III;
(d) absence or presence of phenotype mapped to a Class I marker;
(e) absence or presence of phenotype mapped to a Class II marker;
(f) absence or presence of phenotype mapped to a Class III marker; and
(g) absence or presence of a junction between methylated and unmethylated DNA; and
(h) absence or presence of phenotype mapped to a junction between methylated and unmethylated DNA.
(2) a computer readable medium that stores the code.

Yet another aspect of the invention extends to a computer system for assessing the likely phenotype of a cell based on methylation profile wherein said computer system comprises:
(1) a machine-readable data storage medium comprising a data storage material encoded with machine-readable data, wherein said machine-readable data comprise values for one or more features, wherein said features are selected from:
   (a) absence or presence of AMP marker Class I;
   (b) absence or presence of AMP marker Class II:
   (c) absence or presence of AMP marker Class III;
   (d) absence or presence of phenotype mapped to a Class I marker;
   (e) absence or presence of phenotype mapped to a Class II marker;
   (f) absence or presence of phenotype mapped to a Class III marker;
   (g) absence or presence of a junction between methylated and unmethylated DNA; and
   (h) absence or presence of phenotype mapped to a junction between methylated and unmethylated DNA;
(2) a working memory for storing instructions for processing said machine-readable data;
(3) a central-processing unit coupled to said working memory and to said machine-readable storage medium, for processing said machine-readable data to provide comparison of phenotype and AMP marker classes; and
(4) an output hardware coupled to said central processing unit for receiving said data of comparison.

The present invention further extends to searching for homologous AMP markers, once identified. This then provides a basis for identifying similar control mechanisms in other genes, promoters or regulatory regions. For example, tissue-specific AMP markers or AMP markers associated with a disease are cloned, sequenced and mapped on the genome. Coding sequences adjacent or limited to the mapped AMP marker are identified. Genome-wide microarray analysis is conducted with cDNA probes from different tissues or from normal versus diseased cells to reveal other coding sequences that are coordinately regulated with the coding sequence linked to the said tissue or disease-specific AMP marker. Computer algorithms are then used to identify conserved nucleotide signatures common to all coordinately expressed genes, which in turn are correlated to the methylation status of an AMP marker in the vicinity of each coding sequence. The discovered nucleotide signature can then be used to discover further coordinately regulated coding sequences around the genome regulated by methylation status of an adjacent AMP marker.

A combined AMP analysis approach, or AMP analysis on individuals or tissues of individuals, is adopted to identify DNA methylation markers linked or associated with important phenoypes such as disease, a diseased tissue (e.g. cancer), aging or exposure to a toxin (e.g. excessive alcohol or smoke). For the combined AMP analysis approach, DNA is bulked for normal versus affected individuals or tissues. AMP analysis and comparison between normal and affected DNA bulks identifies candidate diagnostic markers associated with the phenotype. Promising markers identified by this approach are then tested on a large number of normal versus affected individuals or tissues. When a marker is identified to be always or almost always or very often associated with the phenotype, the marker will be cloned and sequenced, and a diagnostic test developed. The AMP is also be mapped on the genome to identify coding or non-coding genes linked to the methylation change in affected individuals or tissues. These linked coding or non-coding genes or the encoded proteins are identified as therapeutic targets for the development of drugs or therapy.

In cases where the gene is methylated and silenced in affected individuals or tissues, compounds are screened in high throughput fashion in stable cell lines or individuals to identify drugs that result in demethylation and reactivation of the affected gene. Alternatively, a normal active copy of the affected gene are transfected as a transgene into cells to correct the defect. Such transgenes are introduced with modulating sequences that protect the transgene from methylation and keep it unmethylated and transcriptionally active.

In cases where the gene is unmethylated and transcriptionally active or transcriptionally over-active in affected individuals or tissues, compounds are screened in high throughput fashion in stable cell lines to identify drugs that result in methylation and silencing of the affected gene. Alternatively, a transgene encoding a double stranded RNA homologous to the affected AMP or homologous to sequences linked to the AMP, or double stranded RNA homologous to the AMP or homologous to sequences linked to the AMP, are transfected as a transgene into cells to methylate the gene, silence it and thereby correct the defect. Such double stranded RNA-encoding transgenes are introduced with modulating sequences which protect it from methylation, keep it transcriptionally active and producing double stranded RNA.

The present invention is further described by the following non-limiting Examples.

### EXAMPLE 1

### AMP Protocol

The following protocol may need to be adapted for use in particular plant or animal cells. However, in general, the procedure involves isolating genomic DNA, subjecting the DNA to HpaII digestion and subjecting the digest to an amplification reaction using a primer which spans all or part of the HpaII recognition sequence, i.e.

```
  ↓
CCGG,
      ↑
```

such that if HpaII has digested the nucleotide sequence, primer extension cannot occur.

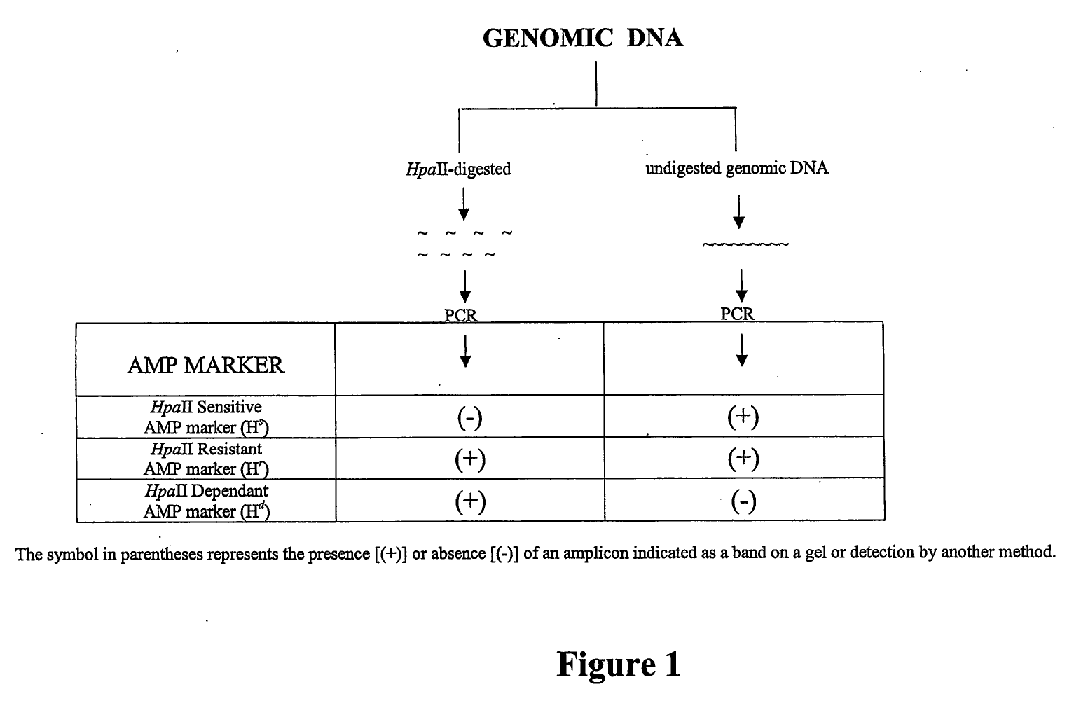

A diagram showing the AMP protocol is shown in FIG. 1.

Three classes of product are expected:

| | |
|---|---|
| Class I | HpaII digestion-resistant (H^{r}) markers, indicative of |
| | methylation |
| Class II | HpaII digestion-sensitive (H^{s}) markers, indicative of non- |
| | methylation. |
| Class III | HpaII digestion-dependent (H^{d}) markers. Amplicon is |
| | methylated or partly methylated and is linked to an |
| | unmethylated HpaII site followed by an inhibitor sequence. |
| | Cleavage of the unmethylated site removes inhibition and |
| | allows amplification. |

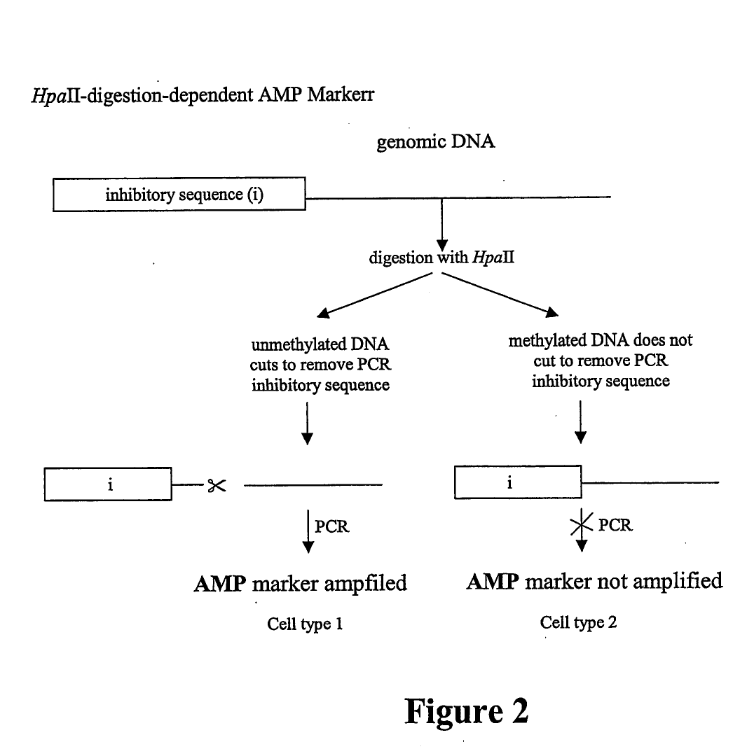

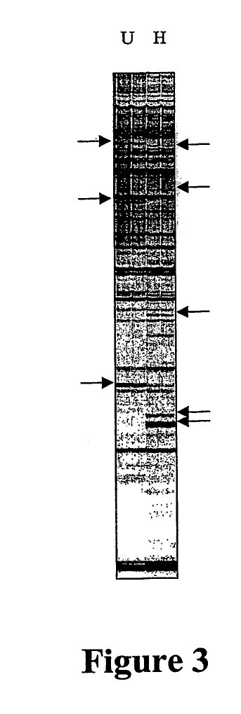

The existence of an H^{d} (Class III) marker is further shown in FIG. 2. An H^{d} marker represents a junction between methylated and unmethylated DNA. FIG. 3 provides an example of the different classes of AMP markers.

The following protocol is useful for most cells such as from humans, sugarcane and wheat.

The DNA marker protocols are based on arbitrarily-primed PCR, using high concentrations (5 μM) of small primers, ten nucleotides in length. PCR is performed with a hot start (85° C.), an initial denaturation at 94° C. for 5 min, followed by 30 cycles of: 94° C. for 30 secs, 60 sec at each of 57° C., 56° C., 55° C., 54° C. and 53° C. and a final extension step at 72° C. for 5 min. Highly reproducible RAFs were amplified from either purified DNA or alkali treated leaf tissue (Klimyuk et al., Plant J. 3: 493-494, 1993) to demonstrate DNA sequence change between sugarcane lines. The amplified DNA methylation polymorphisms (AMP) protocol is similar to the RAF protocol, but it uses genomic DNA digested with methylation sensitive enzymes before it is used as template in PCR using oligonucleotide primers with recognition sequences for the methylation-sensitive enzyme.

The protocol useful for eukaryotic cells such as from humans, sugarcane or wheat is as follows.

The method is a PCR-based DNA marker protocol for detecting AMP markers in genomic DNA. AMP markers are detected by using oligonucleotide primers carrying recognition sequences for methylation-sensitive restriction endonucleases on genomic template digested with the corresponding enzyme. The procedure listed below is for use of the methylation-sensitive enzyme HpaII and corresponding HpaII oligonucleotide primers, but the protocol can be modified accordingly for all other methylation-sensitive enzymes.

Template Preparation:

1. Digest 5.0 μg of high quality genomic DNA with the methylation sensitive enzyme (e.g. HpaII) [overnight digestion is convenient].

Digest Reaction (100 μL)

| | |
|---|---|
| 10 × enzyme buffer | 10 μL |
| HpaII (10 U/μl) | 5 μL |
| DNA | 5 μg |
| sterile dH₂O to final Vol. | 100 μL |

2. Run 250 ng (5 μL) of digest on 0.7% w/v agarose gel to check digestion.

3. Remove 250 ng (5 μL) and keep for later (to compare with recovery after clean-up).

4. Precipitate digested DNA with 0.1 Vol. 3 M Na Acetate (pH 6), 2 Vol. 100% v/v EtOH, 1 μL (20 μg/μl) glycogen. Mix gently, leave on bench for 10 to 15 mins.

5. Microfuge at 4° C. (13000 rpm) 30 mins. Immediately after spinning, remove the supernatant and wash pellet with 70% v/v EtOH. Remove EtOH and dry pellet (2 mins in Speedie Vac or air-dry at room temp in laminar flow).

6. Resuspend pellet in 50 μL sterile dH₂O (ca. 100 ng/μL). To check recovery of the clean-up run 2.5 μL (ca. 250 ng) on gel alongside the 250 ng kept from step 3. Estimate recovery and dilute samples to 50 ng/μL for use in the AMP reaction.

A PCR procedure is then conducted using the following primers:

HpaII Oligonucleotide Primers (Operon)

| | | |
|---|---|---|
| Oligo | 5′-3′ | SEQ ID NO: |

| | | |
|---|---|---|
| B-16 | TTTGCCCGGA | 1 |
| C-08 | TGGACCGGTG | 2 |
| D-20 | ACCCGGTCAC | 3 |
| F-17 | AACCCGGGAA | 4 |
| F-18 | TTCCCGGGTT | 5 |
| I-08 | TTTGCCCGGT | 6 |
| J-01 | CCCGGCATAA | 7 |
| J-14 | CACCCGGATG | 8 |
| M-17 | TCAGTCCGGG | 9 |
| N-09 | TGCCGGCTTG | 10 |
| P-05 | CCCCGGTAAC | 11 |
| V-15 | CAGTGCCGGT | 12 |
| V-17 | ACCGGCTTGT | 13 |
| W-03 | GTCCGGAGTG | 14 |
| W-15 | ACACCGGAAC | 15 |
| AB-16 | CCCGGATGGT | 16 |
| AE-11 | AAGACCGGGA | 17 |
| AF-16 | TCCCGGTGAG | 18 |
| AJ-15 | GAATCCGGCA | 19 |
| AK-18 | ACCCGGAAAC | 20 |
| AM-09 | TGCCGGTTCA | 21 |
| AN-14 | AGCCGGGTAA | 22 |
| AP-20 | CCCGGATACA | 23 |
| AQ-16 | CCCGGAAGAG | 24 |
| AR-18 | CTACCGGCAC | 25 |
| AT-10 | ACCTCCGGTC | 26 |
| AV-14 | CTCCGGATCA | 27 |
| AY-03 | TTTCCGGGAG | 28 |
| BB-09 | AGGCCGGTCA | 29 |
| BB-18 | CAACCGGTCT | 30 |
| BF-13 | CCGCCGGTAA | 31 |
| BG-17 | TCCGGGACTC | 32 |

Useful PCR stock solutions include:

1. 10× DAF buffer=100 mM Tris pH 8, 100 mM KCl, 50 mM MgCl₂;

2. 1 mM dNTPs;

3. α-labelled ³³P-dATP, 10 μCi/μL (as from supplier); and

4. 10 μM 10-mer primer stocks.

The PCR procedure is conducted as follows:

PCR is conducted in a 10 μL volume, with a single oglionucleotide primer, 10 nucleotides long (see above).

| Reaction Mix | 1 reaction | 10 reactions | Final concentration |
|---|---|---|---|
| Stoffel fragment 10 U/μL | 0.15 μL | 1.5 μL | 1.5 U/10 μL |
| 10 × DAF buffer | 1.0 μL | 10.0 μL | |
| dNTPs (1 mM) | 0.2 μL | 2.0 μL | 20 μM |
| Sterile dH₂O | 2.4 μL | 24.0 μL | |
| α-labeled ³³P-dATP | 0.25 μL | 2.5 μL | 2.5 μCi/10 μL |
| (10 μCi/μL) | | | |
| Total | 4.0 μL | 40.0 μL | |

Aliquot into each PCR tube 4 μL of reaction mix, then add 5 μL primer (final concentration 5 μM) and 1 μl 50 ng/μL digested or genomic DNA.

Prepare reactions on ice and load tubes into the PCR machine with the block at 85° C. (i.e. hot start).

The following conditions are employed:

| | | |
|---|---|---|
| 1. | 94° C. | 5 min |
| 2. | 94° C. | 30 sec |
| 3. | 57° C. | 1 min |
| 4. | 56° C. | 1 min |
| 5. | 55° C. | 1 min |
| 6. | 54° C. | 1 min |
| 7. | 53° C. | 1 min |
| 8. | go to step (2) | 29 more times |
| 9. | 72° C. | 5 min |
| 10. | end. | |

The protocol was slightly modified for the large wheat genome, in that the annealing/extension temperatures were 2° C. higher, i.e.
30 cycles of 94° C. for 5 min
94° C. for 30 sec
59° C. for 1 min
58° C. for 1 min
57° C. for 1 min
56° C. for 1 min
55° C. for 1 min.

Furthermore, the following changes were also made:

1. Add 10 μL of 2× loading buffer (0.0% w/v Bromophenol Blue, 0.05% Xylene Cyanol, 98% v/v formamide, 10 mM EDTA, pH 8) to each PCR sample, mix gently. Before gel electrophoresis, denature PCR reactions at 90° C. for 3 mins, cool on ice.

2. Separate the products on sequencing gels (4% w/v polyacrylamide gel, 7.5 M urea in 1×TBE, run at 100 W, 2 hr 15 mins). Dry gel and expose to film for 6 hours or overnight.

The extent of DNA methylation change was determined in 24 sugarcane lines cloned by tissue culture. All 24 lines investigated had widespread DNA methylation changes and unique AMP profiles. An average of 0.8% DNA methylation polymorphism over more than 1,000 markers was observed between the parent variety and somaclones and both increases and decreases in DNA methylation were observed. DNA methylation polymorphism was also demonstrated in conventionally propagated plants, but the level was about two-fold greater (p<0.05) in tissue culture propagated plants.

Clearly, DNA methylation change predisposed sugarcane clones to genome instability as about 20% of non-transgenic somaclones lost DNA as measured by randomly amplified DNA fingerprinting protocols (RAFs) and this finding was confirmed with simple sequence repeat (SSR) markers. Up to 2% of RAF markers were lost in a selection of transgenic lines of sugarcane. Genetic mapping of DNA markers susceptible to loss showed that they were often deleted as a linkage group. The inventors detected an example of chromosome breakage as distinct from chromosome loss in one transgenic line. Interestingly, there was a significant correlation between loss of markers and reduced yield in preliminary field trials on transgenic lines (p=0.017).

This example showed that a minimal passage through tissue culture altered the methylation status of about 1% of the sugarcane genome, as randomly detected by the AMP technology. Transposon activity can lead to chromosome loss and/or breakage (e.g., see English et al., Plant Cell 5: 501-514, 1993). Thus, the increased DNA methylation change in somaclones as shown here may have direct effects on gene expression as well as predisposing the sugarcane genome to instability. The AMP technologies in accordance with the present invention may usefully be applied as diagnostic tests to identify tissue culture conditions that minimize DNA methylation change and loss of DNA during the tissue culture phase of genetic engineering of plants and animals.

### EXAMPLE 2

### Detection of DNA Polymorphisms in Human Body Fluid

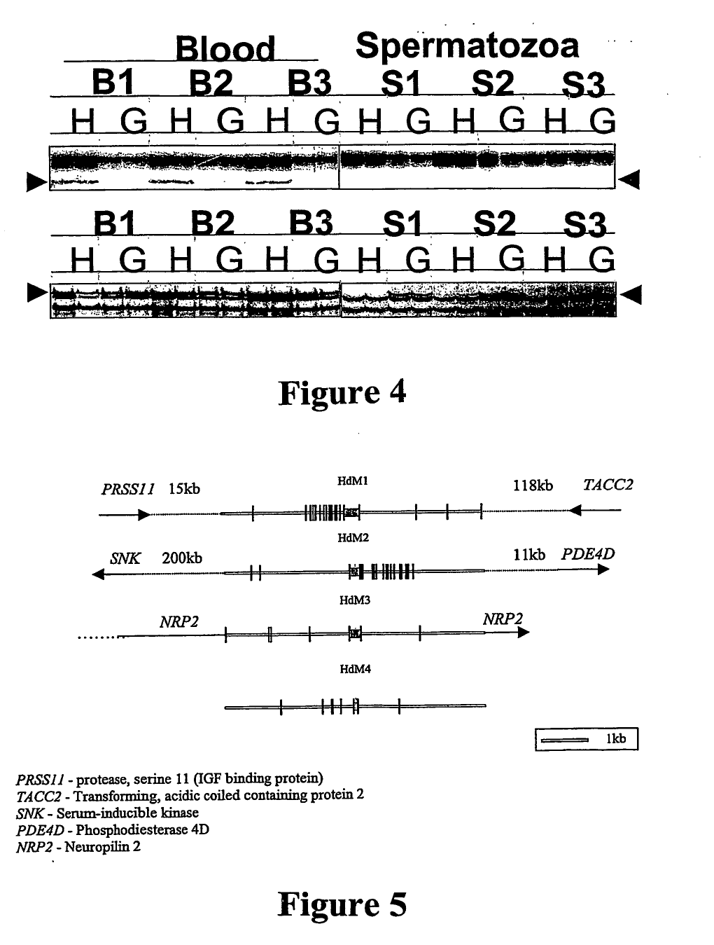

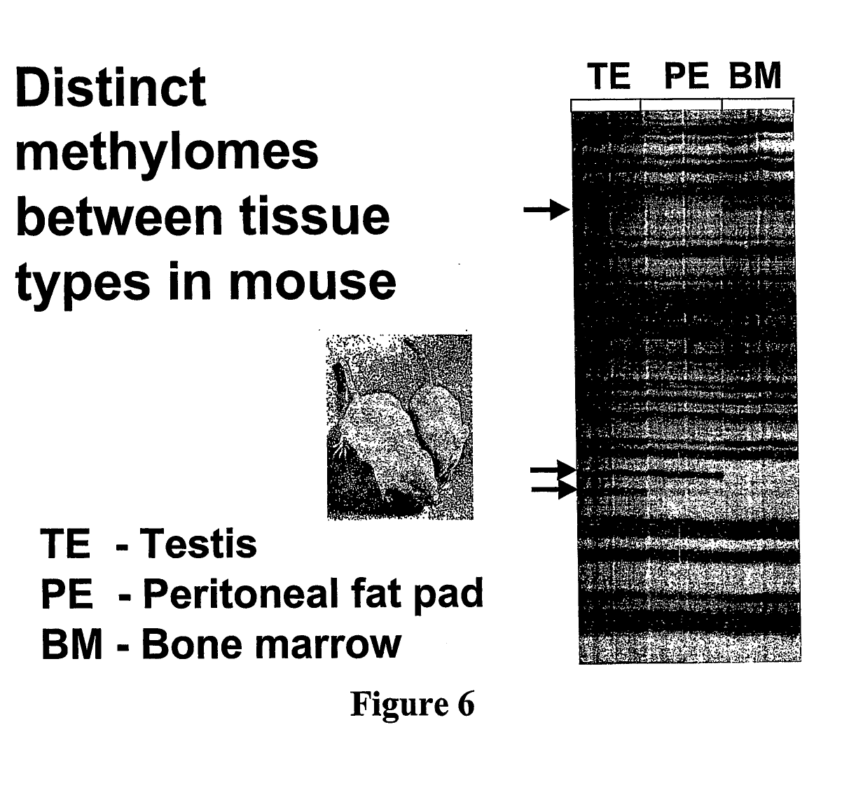

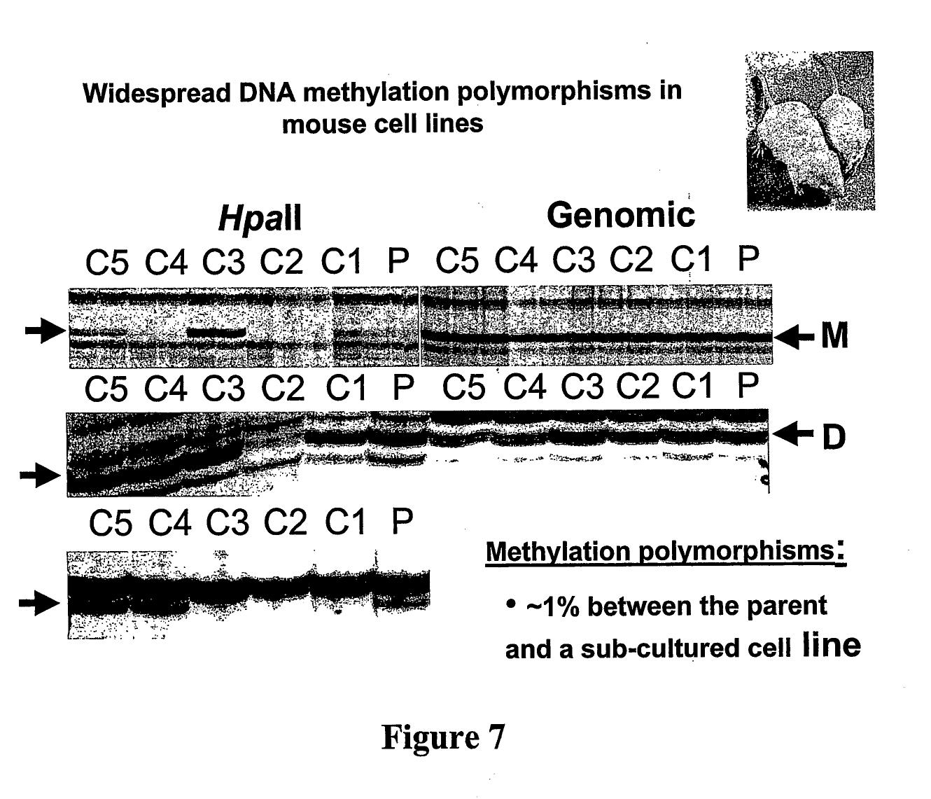

Using the AMP technology, abundant and distinct tissue-specific DNA methylation polymorphisms were readily detected between human blood and sperm (FIG. 4). Most of the tissue specific DNA methylation polymorphisms were Class III digestion dependent markers and surprisingly, 30% of these were polymorphic in methylation status between these tissues. Between five blood samples and three sperm samples (from eight separate individuals), 80% of variation in AMP profiles generated from HapII-digested template could be explained by the tissue type, and the remaining 20% of variation could not be distinguished from nucleotide (rather than DNA methylation) polymorphism.

In another example, blood from three sets of young, healthy, identical twins were also assessed for DNA methylation polymorphisms over 8,000 CpG sites in the genome, representing an average of about 340 CpG sites per human chromosome. There was 100% concordance of AMP profiles of identical twins thereby indicating stringent genetic programming of DNA methylation patterns in human cells to produce cell-specific “methylomes”. Interestingly, an AMP marker was detected that showed an age-dependent methylation change. This has implications in targets for therapy of age-related conditions or illnesses and in forensic medicine.

### EXAMPLE 3

### Identification of Class III Markers in Human Genome

FIG. 5 is a diagrammatic representation showing map location of four human digestion-dependent Class III AMP markers and the flanking HpaII sites. Each of the digestion-dependent markers that was cloned and sequenced was 100% homologous to sequenced regions of the human genome. The positions of the Class III amplicons (HdM 1-4) [SEQ ID NOS:33-36] are indicated by thick shaded boxes on the chromosomal segment. The locations of HpaII sites are represented by vertical lines and the distance to, and orientation of nearby genes are indicated. Southern analysis has shown that Class III markers represent the junction between methylated and unmethylated DNA in the genome. Tissue-specific Class III AMP markers often map to the CpG islands and often represent tissue-specific modification of methylation in or around CpG islands.

### EXAMPLE 4

### Identification of Promoter Elements that Contain CpG Islands in Human Genomic DNA

Human promoter elements that contains CpG islands are identified by searching the human genome database and selecting promoter element sequences that comprise regions of DNA which are approximately 180 bp in length and that have a sequence complexity comprising a G+C content above 50%. An example of such a promoter element comprising a CpG island is rpA2 promoter element (shown below):

[image]

[image]

The CpG islands present in the promoter element are highlighted. The rpA2 promoter element contains two CpG islands, which are highlighted. CpG islands in this promoter element indicate that the expression of the gene is regulated by the methylation status of the promoter element.

### EXAMPLE 5

### Detection of Promoter Elements that Contain CpG Islands in Plant Genomic DNA

A plant promoter element that contains CpG islands is identified by searching plant genome databases and selecting promoter element sequences that comprise regions of DNA which are at least 180 bp in length and that have a sequence complexity comprising a G+C content above 50%. An example of such a promoter element comprising a CpG island is the rbp21 promoter element derived from the rbp21 gene from Oryza sativa (shown below):

| [SEQ ID NO:38] | |
|---|---|
| ctgcagagag gatgaccctt gtcaccaccg tcatgtacga ggctgcttca ccactgcctc | 60 |
| actgccacca gcgtctcccg ccgcgtgcaa tacaagaaga aacatcgaac ggtcatataa | 120 |
| ggtaagaccc actaccgatt taacctatca ttcccacaat ctaatccact tatttctctt | 180 |
| cccatgatct tatcctctca tttctcctca ctacttttgc atttgtagga aacacaatga | 240 |
| caccgtcgaa gaaagctggt ggagcaccgt agccagcaat caccaaaaca cagaggggag | 300 |
| gaggtcggca gcggccatgc ggacggcgat gagacaacgc gacgcaaaga gggaggagga | 360 |
| cgttggcgat catgctggtg ttggcggagg aggtcactgg ccatgcgaat gacagcgggg | 420 |
| cagcgcaaca caaaaagggg ggaggatgcc ggcgaccacg ctagtaccat gaagcaagat | 480 |
| gatgtgaaag ggaggaccgg acgagggttg gacctctgcc gccgacgtga agagcgtgat | 540 |
| gtgtagaagg agatgttaga ccagatgccg acgcaactta gccctgcaag tcacccgact | 600 |
| gcatatcgct gcttgccctc gtcctcatgt acacaatcag cttgcttatc tctccatact | 660 |
| tgtcgtttgt ttcccgtggc cgaaatagaa gaagacagag gtgggttttg ttggagagtt | 720 |
| ttagtggtat tgtaggccta tttgtaattt tgttgtactt tattgtatta atcaataaag | 780 |
| gtgtttcatt ctattttgac tcaatgttga atccattgat ctcttggtgt tgcactcagt | 840 |
| atgttagaat attcattccg ttgaaacaat cttggttaag ggttggaaca tttttatctg | 900 |
| ttcggtgaaa catccgtaat attttcgttg aaacaatttt tatccgacag caccgtccaa | 960 |
| caatttacac caatttggac gtgtgataca tagcagtccc caagtgaaac tgaccaccag | 1020 |
| ttgaaaggta tacaaagtga acttattcat ctaaaagacc gcagagatgg gccgtggccg | 1080 |
| tggctgcgaa acgacagcgt tcaggcccat gagccattta ttttttaaaa aaatatttca | 1140 |
| acaaaaaaga gaacggataa aatccatcga aaaaaaaaaa ctttcctacg catcctctcc | 1200 |
| tatctccatc cacggcgagc actcatccaa accgtccatc cacgcgcaca gtacacacac | 1260 |
| atagttatcg tctctccccc cgatgagtca ccacccgtgt cttcgagaaa cgcctcgccc | 1320 |
| gacaccgtac gtgcgccacc gccgcgcctg ccgcctggac acgtccggct cctctcccgc | 1380 |
| cgcgctggcc accgtccacc ggctcccgca cacgtctccc tgtctccctc cacccatgcc | 1440 |
| gtggcaatcg agctcatctc ctcgcctcct ccggcttata aatggcggcc accaccttca | 1500 |
| cctgcttgca | |

The CpG islands present in the promoter element are highlighted. The rbp21 promoter element contains two CpG islands. The presence of a CpG island indicates that the activity of the promoter element is regulated by methylation.

### EXAMPLE 6

### Use of AMP to Assess Stable Mammalian Cell Lines

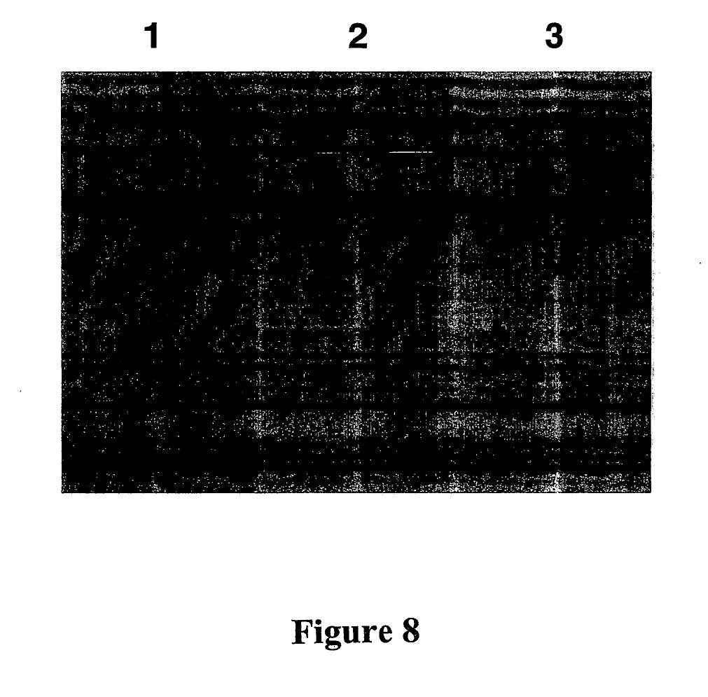

Mammalian cell lines are used for many applications in medical research. It is important in many applications that cell lines used are stable in their phenotypes. AMP is used to identify stable versus unstable mammalian cell lines. AMP has been used to demonstrate that subcultures of the human colon carcinoma cell line HCT116 are very stable and identical in terms of DNA methylation and DNA sequence (FIG. 8). Similarly, AMP can be used to monitor DNA methylation and genome stability in cloned animals.

### EXAMPLE 7

### Use of AMP to Assess Stable Cell Lines

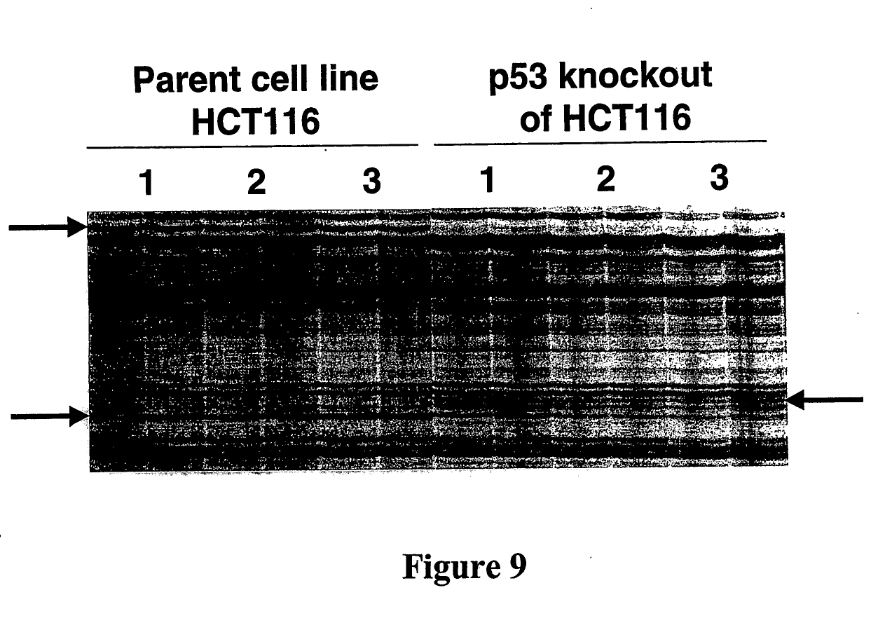

Cell lines that are stable in terms of DNA methylation and DNA sequence can be used with the AMP protocol to identify and discover genes involved in control of DNA methylation and genome stability, or to identify processes that destabilize DNA methylation and genome integrity. AMP has been used to demonstrate that knocking out the tumor suppressor gene p53 in the human colon carcinoma cell line HCT116 results in the induction of about 3% DNA methylation and 0.3% DNA sequence change to the genome as measured by AMP (FIG. 9). These genomic changes could be due to loss of p53 function (indicating a key role for p53 in control of DNA methylation and maintenance of genome stability) or to the physical process of transfecting the cell line. Thus AMP can be used to identify and discover genes involved in control of DNA methylation and genome stability, and to identify processes or toxins that destabilize DNA methylation and genome integrity in mammals.

### EXAMPLE 8

Detection of DNA Methylation Polymorphisms in Genomic DNA

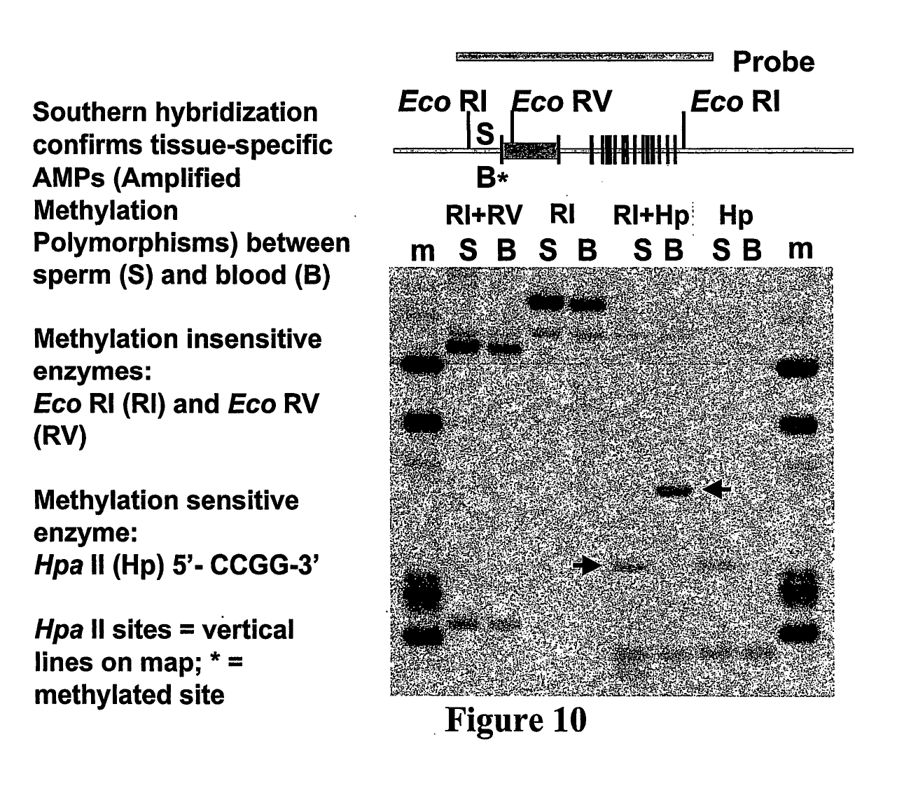

Southern analysis confirms that AMP detects DNA methylation polymorphisms in genomic DNA and the junctions of methylated and unmethylated DNA in the genome (FIG. 10).

### EXAMPLE 9

### Combined AMP Analysis

DNA from normal or abnormal tissue (e.g. cancer) is separately bulked. AMP analysis is conducted to compare DNA from the normal and abnormal tissue. Candidate fragments present or absent in abnormal tissue relative to normal tissue are then cloned and analyzed for subsequent development as a potential diagnostic marker for disease and/or as a potential therapeutic product itself or as a target for antagonists and agonists. In addition, methylation- or demethylation-facilitating sequences may be used to methylate or demethylate target DNA.

### EXAMPLE 10

### Use of Substractive Hybridization to Enrich for HpaII Dependent Markers

With comparing genomes subjected to AMP analysis, given the large number of bands which are present, it is possible that HpaII digestion-dependent markers are masked by other bands. Subtractive amplification is used to increase the number of HpaII-digestion dependent PCR detected in each PCR.

The PCR-Select cDNA subtraction kit (Clontech, Palo Alto, USA) is conveniently used for the subtractive hybridization.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### Bibliography

Baylin, S. B. and Herman, J. G. (2000) Trends Genet. 16: 168-174.
Bentley, S. and Bassam, B. J. (1996). A robust DNA amplification fingerprinting system applied to analysis of genetic variation within Fusarium oxysporum fsp cubense. J. Phytopath. 14: 207-214.
Bonner and Laskey (1974) Eur. J. Biochem. 46: 83.
English, J., Harrison, K. and Jones, J. D. (1993). A genetic analysis of DNA sequence requirements for Dissociation state I activity in tobacco. Plant Cell 5: 501-14.
Hendrich, B. (2000) Current Biology 10: 60-63.
Herman, J. G. (1999) Semin. Cancer Biol. 9: 359-67.
Klimyuk, V. I., Carroll, B. J., Thomas, C. M and Jones, J. D. (1993). Alkali treatment for rapid preparation of plant material for reliable PCR analysis. Plant J. 3: 493-494.
Laird, P. W. and Jaenisch, R. (1996) Annu. Rev. Genet. 30: 441-464.
Lengauer, C. et al. (1997) Proc. Natl. Acad. Sci. USA. 94: 2545-2550.
Marmur and Doty (1962) J. Mol. Biol. 5: 109.
O'Neill, R. J. W. et al. (1998) Nature 393: 68-72.
Walsh, C. P. and Bestor, T. H. (1999) Genes Dev. 13: 26-34.
Yoder, J. A. et al. (1997) Trends Genet. 13: 335-340.

1. A method for determining the methylation profile of one or more nucleotides at one or more sites within the genome of a eukaryotic cell or group of cells. said method comprising obtaining a sample of genomic DNA from said cell or group of cells, digesting a sub-sample of said sample of genomic DNA with a methylation-sensitive enzyme having a recognition nucleotide sequence comprising a nucleotide sequence capable of methylation at one or more of the above-mentioned sites, subjecting said digested DNA to an amplification reaction using primers comprising a nucleotide sequence capable of annealing to a non-cleaved form of a cleavable nucleotide sequence of the methylation-sensitive enzyme recognition sequence and subjecting the products of the amplification to separation or other detection means relative to a control, said control comprising another sub-sample of said sample of genomic DNA not subjected to digestion by the methylation-sensitive enzyme but subjected to an amplification reaction using the same primers as for the digested DNA sample and then subjecting the products of this amplification reaction to said separation or other detection means, wherein the presence of the same amplification products in enzyme digested and non-digested samples is indicative of a methylation-sensitive enzyme-digestion-resistant marker, the absence and presence of amplification products in enzyme digested and undigested samples, respectively, is indicative of a methylation-sensitive enzyme-digestion-sensitive markers, and the presence and absence of amplification products in enzyme digested and undigested samples, respectively, is indicative of a methylation-sensitive enzyme-digestion-dependent marker.

2. The method of claim 1 wherein the junctions of methylated and unmethylated DNA are detected.

3. The method of claim 1 wherein the potentially-methylated sites are CpG or CpNpG nucleotides within the recognition sequence of the methylation-sensitive enzyme.

4. The method of claim 1 wherein the methylation-sensitive enzyme is selected from the list comprising AatII, AciI, AclI, AgeI, AscI, AvaI, BamHI, BsaAI, BsaH1, BsiE, BsiW, BsrF, BssHII, BstBI, BstUI, Cla1, EagI, HaeII, HgaI, HhaI, HinPI, HpaII, MloI, MspI, NaeI, NarI, NotI, NruI and PmlI.

5. The method of claim 4 wherein the methylation-sensitive enzyme is HpaII or its functional equivalent.

6. The method of claim 5 wherein the methylation-sensitive enzyme is HpaII.

7. The method of claim 1 wherein the amplification reaction is selected from polymerase chain reaction (PCR), ligase chain reaction (LCR), gap filling LCR (GLCR), Qβ replicase; stand displacement amplification (SDA); self-sustained sequence replication (3SR) nucleic acid sequence-based amplification (NASBA).

8. The method of claim 7 wherein the amplification reaction is PCR.

9. The method of claim 8 wherein the amplification reaction is real-time PCR.

10. The method of claim 1 wherein the separation means is gel electrophoresis.

11. The method of claim 1 wherein the eukaryotic cell is a human or mammalian embryonic stem (ES) cell, embryonic germ (EG) cell, post-natal stem cell, committed cell or mature cell or a cell having a state of development in between any of the above cells.

12. The method of claim 1 wherein the eukaryotic cell is a cancer cell or cancer

13. The method of claim 1 wherein the cell is a plant cell.

14. The method of claim 1 wherein the cell is a non-mammalian animal cell.

15. The method of claim 1 wherein the cell is a mammalian cell.

16. A method for determining the methylation profile of one or more CpG or CpNpG nucleotides at one or more sites within the genome of a eukaryotic, cell or group of cells, said method comprising obtaining a sample of genomic DNA from said cell, digesting a sub-sample of said sample of genomic DNA with HpaII which has a recognition nucleotide sequence corresponding to or within said sites, subjecting said digested DNA to an amplification reaction using primers comprising a nucleotide sequence capable of annealing to a non-cleaved form of a HpaII cleavable nucleotide sequence and subjecting the products of the amplification reaction to separation or other detection means relative to a control but subjected to an amplification reaction using the same primers as for the digested DNA sample and then subjecting the products of the amplification reaction to the separation or other detection means, said control comprising another sub-sample of said sample of genomic DNA not subjected to digestion by HpaII, wherein the presence of amplification products in enzyme digested and non-digested samples is indicative of a HpaII-digestion-resistant marker (H^{I}), the absence and presence of amplification products in enzyme digested and undigested samples, respectively, is indicative of a HpaII-digestion-sensitive marker (H^{S}) and the presence and absence of amplification products in enzyme digested and undigested samples, respectively, is indicative of a HpaII-digestion-dependent marker (H^{d}).

17. The method of claim 16 wherein the junctions of methylated and unmethylated DNA are detected.

18. The method of claim 16 wherein the amplification reaction is PCR.

19. The method of claim 18 wherein the amplification reaction is real-time PCR.

20. The method of claim 16 wherein the eukaryotic cell is a human or mammalian embryonic stem (ES) cell, embryonic germ (EG) cell, post-natal stem cell, committed cell or mature cell or a cell having a state of development in between any of the above cells.

21. The method of claim 16 wherein the eukaryotic cell is a cancer cell or a cancer cell line.

22. The method of claim 16 wherein the cell is a plant cell.

23. The method of claim 16 wherein the cell is a non-mammalian animal cell.

24. The method of claim 14 wherein the cell is a mammalian cell.

25. A methylation profile of the genome of a eukaryotic cell or group of cells, the methylation profile comprising the presence or absence of methylation at particular sites and/or junctions between methylated and unmethylated regions, said method comprising obtaining a sample of genomic DNA from said cell or group of cells, digesting a sub-sample of said sample of genomic DNA with a methylation-sensitive enzyme having a recognition nucleotide sequence comprising a nucleotide capable of methylation at one or more of the above-mentioned sites, subjecting said digested DNA to an amplification reaction using primers comprising a nucleotide sequence capable of annealing to a non-cleaved form of a cleavable nucleotide sequence of the methylation-sensitive enzyme recognition sequence and subjecting the products of the amplification reaction to separation or other detection means relative to a control but subjected to an amplification reaction using the same primers as for the digested DNA sample and then subjecting the products of the amplification reaction to the separation or other detection means, said control comprising another sub-sample of said sample of genomic DNA not subjected to digestion by the methylation-sensitive enzyme wherein the presence of amplification products in enzyme digested and non-digested samples is indicative of a methylation-sensitive enzyme-digestion-resistant marker, the absence and presence of amplification products in enzyme digested and undigested samples, respectively, is indicative of a methylation-sensitive enzyme-digestion-sensitive marker and the presence and absence of amplification products in enzyme digested and undigested samples, respectively, is indicative of a methylation-sensitive enzyme-digestion-dependent marker.

26. The method of claim 25 wherein the junctions of methylated and unmethylated DNA are detected.

27. The method of claim 25 wherein the potentially-methylated sites are CpG or CpNpG nucleotides within the recognition sequence of the methylation-sensitive enzyme.

28. The method of claim 25 wherein the methylation-sensitive enzyme is selected from the list comprising AatII, AciI, AclI, AgeI, AscI, AvaI, BamHI, BsaAI, BsaH1, BsiE, BsiW, BsrF, BssHII, BstBI, BstUI, Cla1, EagI, HaeII, HgaI, HhaI, HinPI, HpaII, MloI, MspI, NaeI, NarI, NotI, NruI and PmlI.

29. The method of claim 28 wherein the methylation-sensitive enzyme is HpaII or its functional equivalent.

30. The method of claim 29 wherein the methylation-sensitive enzyme is HpaII.

31. The method of claim 25 wherein the amplification reaction is selected from polymerase chain reaction (PCR), ligase chain reaction (LCR), gap filling LCR (GLCR), Qβ replicase; stand displacement amplification (SDA); self-sustained sequence replication (3SR) nucleic acid sequence-based amplification (NASBA).

32. The method of claim 31 wherein the amplification reaction is PCR.

33. The method of claim 32 wherein the amplification reaction is real-time PCR.

34. The method of claim 25 wherein the separation means is gel electrophoresis.

35. The method of claim 25 wherein the eukaryotic cell is a human or mammalian embryonic stem (ES) cell, embryonic germ (EG) cell, post-natal stem cell, committed cell, or mature cell or a cell having a state of development in between any of the above cells.

36. The method of claim 25 wherein the eukaryotic cell is a cancer cell or cancer cell line.

37. The method of claim 25 wherein the cell is a plant cell.

38. The method of claim 25 wherein the cell is a non-mammalian animal cell.

39. The method of claim 21 wherein the cell is a mammalian cell.

40. An assay device in the form of a kit useful for determining the methylation profile of one or more nucleotides at one or more sites within the genome of a eukaryotic cell, said kit comprising in compartmental form multiple compartments each adapted to comprise one or more of buffers, diluents and enyzmes as single or multiple components which are optionally required to be admixed prior to use, said kit further comprising instructions for use wherein the method is conducted by obtaining a sample of genomic DNA from said cell, digesting a sub-sample of said sample of genomic DNA with a methylation-sensitive enzyme having a recognition nucleotide sequence comprising a nucleotide capable of being methylated at one or more of the above-mentioned sites, subjecting said digested DNA to an amplification reaction using primers comprising a nucleotide sequence capable of annealing to a non-cleaved form of a cleavable nucleotide sequence of the methylation-sensitive enzyme recognition sequence and subjecting the products of the amplification reaction to separation or detection means relative to a control but subjected to an amplification reaction using the same primers as for the digested DNA sample and then subjecting the products of the amplification reaction to the separation or other detection means, said control comprising another sub-sample of said sample of genomic DNA not subjected to digestion by the methylation-sensitive enzyme, wherein the presence of amplification products in enzyme digested and non-digested samples is indicative of a methylation-sensitive enzyme-digestion-resistant marker) and wherein the absence and presence of amplification products in enzyme digested and undigested samples, respectively, is indicative of a methylation-sensitive enzyme-digestion-sensitive marker and wherein the presence and absence of amplification products in enzyme digested and undigested samples, respectively, is indicative of a methylation-sensitive enzyme-digestion-dependent marker.

41. The kit of claim 40 wherein the junctions of methylation and unmethylated DNA are detected.

42. The kit of claim 40 wherein the potentially-methylated sites are CpG or CpNpG nucleotides within the recognition sequence of the methylation-sensitive enzyme.

43. The kit of claim 40 wherein the methylation-sensitive enzyme is selected from the list comprising AatII, AciI, AclI, AgeI, AscI, AvaI, BamHI, BsaAI, BsaH1, BsiE, BsiW, BsrF, BssHII, BstBI, BstUI, ClaI, EagI, HaeII, HgaI, HhaI, HinPI, HpaII, MloI, MspI, NaeI, NarI, NotI, NruI and PmlI.

44. The kit of claim 43 wherein the methylation-sensitive enzyme is HpaII or its functional equivalent.

45. The kit of claim 44 wherein the methylation-sensitive enzyme is HpaII.

46. The kit of claim 40 wherein the amplification reaction is selected from polymerase chain reaction (PCR), ligase chain reaction (LCR), gap filling LCR (GLCR), Qβ replicase; stand displacement amplification (SDA); self-sustained sequence replication (3SR) nucleic acid sequence-based amplification (NASBA).

47. The kit of claim 46 wherein the amplification reaction is PCR.

48. The kit of claim 47 wherein the amplification reaction is real-time PCR.

49. The kit of claim 40 wherein the separation means is gel electrophoresis.

50. The kit of claim 40 wherein the euaryotic cell is a human or mammalian embryonic stem (ES) cell, embryonic germ (EG) cell, post-natal stem cell, committed cell or mature cell or a cell having a state of development in between any of the above cells.

51. The kit of claim 40 wherein the eukaryotic cell is a cancer cell or cancer cell line.

52. The kit of claim 40 wherein the cell is a plant cell.

53. The kit of claim 40 wherein the cell is a non-mammalian animal cell.

54. The kit of claim 40 which detects the junctions of methylated and unmethylated DNA in cells.

55. A method for detecting a change in a cell's or group of cell's developmental state or a cell's or group of cell's exposure to an internal or external stimulus, said method comprising detecting a change in methylation profile over time by the method comprising obtaining a sample of genomic DNA from said cell, digesting a sub-sample of said sample of genomic DNA with a methylation-sensitive enzyme having a recognition nucleotide sequence comprising a nucleotide capable of being methylated at one or more of the above-mentioned sites, subjecting said digested DNA to an amplification reaction using primers comprising a nucleotide sequence capable of annealing to a non-cleaved form of a cleavable nucleotide sequence of the methylation-sensitive enzyme recognition sequence and subjecting the products of the amplification reaction to separation or other detection means relative to a control but subjected to an amplification reaction using the same primers as for the digested DNA sample and then subjecting the products of the amplification reaction to the separation or other detection means, said control comprising another sub-sample of said sample of genomic DNA not subjected to digestion by the methylation-sensitive enzyme, wherein the presence of amplification products in enzyme digested and non-digested samples is indicative of a methylation-sensitive enzyme digestion-resistant marker, and wherein the absence and presence of amplification products in enzyme digested and undigested samples, respectively, is indicative of a methylation-sensitive enzyme-digestion-sensitive marker and wherein the presence and absence of amplification products in enzyme digested and undigested samples, respectively, is indicative of a methylation-sensitive enzyme-digestion-dependent marker.

56. The method of claim 55 wherein the junctions of methylated and unmethylated DNA are detected.

57. The method of claim 55 wherein the potentially-methylated sites are CpG or CpNpG nucleotides within the recognition sequence of the methylation-sensitive enzyme.

58. The method of claim 55 wherein the methylation-sensitive enzyme is selected from the list comprising AatII, AciI, AclI, AgeI, AscI, AvaI, BamHI, BsaAI, BsaHI, BsiE, BsiW, BsrF, BssHII, BstBI, BstUI, ClaI, EagI, HaeII, HgaI, HhaI, HinPI, HpaII, MloI, MspI, NaeI, NarI, NotI, NruI and PmlI.

59. The method of claim 58 wherein the methylation-sensitive enzyme is HpaII or its functional equivalent.

60. The method of claim 59 wherein the methylation-sensitive enzyme is HpaII.

61. The method of claim 55 wherein the amplification reaction is selected from polymerase chain reaction (PCR), ligase chain reaction (LCR), gap filling LCR (GLCR), Qβ replicase; stand displacement amplification (SDA); self-sustained sequence replication (3SR) nucleic acid sequence-based amplification (NASBA).

62. The method of claim 61 wherein the amplification reaction is PCR.

63. The method of claim 62 wherein the amplification reaction is real-time PCR.

64. The method of claim 61 wherein the separation means is gel electrophoresis.

65. The method of claim 61 wherein the eukaryotic cell is a human or mammalian embryonic stem (ES) cell, embryonic germ (EG) cell, post-natal stem cell, committed cell or mature cell or a cell having a state of development in between any of the above cells.

66. The method of claim 61 wherein the eukaryotic cell is a cancer cell or cancer cell line.

67. The method of claim 16 wherein the cell is a plant cell.

68. The method of claim 61 wherein the cell is a non-mammalian animal cell.

69. A computer program product for assessing the likely phenotype of a cell based on methylome profile, said product comprising:
(1) code that receives an input value for one or more of features wherein said features are selected from:
(a) absence or presence of AMP marker Class I;
(b) absence or presence of AMP marker Class II:
(c) absence or presence of AMP marker Class III;
(d) absence or presence of phenotype mapped to a Class I marker;
(e) absence or presence of phenotype mapped to a Class II marker;
(f) absence or presence of phenotype mapped to a Class III marker;
(g) absence or presence of a junction between methylated and unmethylated DNA; and
(h) absence or presence of phenotype mapped to a junction between methylated and unmethylated DNA;
(2) a computer readable medium that stores the code.

70. A computer system for assessing the likely phenotype of a cell based on methylation profile wherein said computer system comprise:
(1) a machine-readable data storage medium comprising a data storage material encoded with machine-readable data, wherein said machine-readable data comprise values for one or more features, wherein said features are selected from:
(a) absence or presence of AMP marker Class I;
(b) absence or presence of AMP marker Class II:
(c) absence or presence of AMP marker Class III;
(d) absence or presence of phenotype mapped to a Class I marker;
(e) absence or presence of phenotype mapped to a Class II marker;
(f) absence or presence of phenotype mapped to a Class III marker;
(g) absence or presence of a junction between methylated and unmethylated DNA; and
(h) absence or presence of phenotype mapped to a junction between methylated and unmethylated DNA;
(2) a working memory for storing instructions for processing said machine-readable data;
(3) a central-processing unit coupled to said working memory and to said machine-readable storage medium, for processing said machine-readable data to provide comparison of phenotype and AMP marker classes; and
(4) an output hardware coupled to said central processing unit for receiving said data of comparison.

71. An isolated stem cell or stem cell line or culture of stem cells characterized as having a methylation profile is determined by the method of claim 1.

72. A method for identifying a genetic sequence associated with a disease phenotype or unwanted trait, said method comprising determining the methylation profile within the genomes of normal compared to diseased tissue by obtaining a sample of genomic DNA from said cell or group of cells, digesting a sub-sample of said sample of genomic DNA with a methylation-sensitive enzyme having a recognition nucleotide sequence comprising a nucleotide sequence capable of methylation at one or more of the above-mentioned sites, subjecting said digested DNA to an amplification reaction using primers comprising a nucleotide sequence capable of annealing to a non-cleaved form of a cleavable nucleotide sequence of the methylation-sensitive enzyme recognition sequence and subjecting the products of the amplification to separation or other detection means relative to a control, said control comprising another sub-sample of said sample of genomic DNA not subjected to digestion by the methylation-sensitive enzyme but subjected to an amplification reaction using the same primers as for the digested DNA sample and then subjecting the products of this amplification reaction to said separation or other detection means, wherein the presence of the same amplification products in enzyme digested and non-digested samples is indicative of a methylation-sensitive enzyme-digestion-resistant marker, the absence and presence of amplification products in enzyme digested and undigested samples, respectively, is indicative of a methylation-sensitive enzyme-digestion-sensitive markers, and the presence and absence of amplification products in enzyme digested and undigested samples, respectively, is indicative of a methylation-sensitive enzyme-digestion-dependent marker wherein the presence of particular markers is indicative of normal or diseased tissue.

73. The method of claim 72 wherein the potentially-methylated sites are CpG or CpNpG nucleotides within the recognition sequence of the methylation-sensitive enzyme.

74. The method of claim 72 wherein the methylation-sensitive enzyme is selected from the list comprising AatII, AciI, AclI, AgeI, AscI, AvaI, BamHI, BsaA1, BsaHI, BsiE, BsiW, BsrF, BssHII, BstBI, BstUI, ClaI, EagI, HaeII, HgaI, HhaI, HinPI, HpaII, MloI, MspI, NaeI, NarI, NotI, NruI and PmII.

75. The method of claim 74 wherein the methylation-sensitive enzyme is HpaII or its functional equivalent.

76. The method of claim 75 wherein the methylation-sensitive enzyme is HpaII.

77. The method of claim 72 wherein the amplification reaction is selected from polymerase chain reaction (PCR), ligase chain reaction (LCR), gap filling LCR (GLCR), Qβ replicase; stand displacement amplification (SDA); self-sustained sequence replication (3SR) nucleic acid sequence-based amplification (NASBA).

78. The method of claim 77 wherein the amplification reaction is PCR.

79. The method of claim 78 wherein the amplification reaction is real-time PCR.

80. A method of treating a subject having a disease state associated with silencing of a genetic sequence as determined by the method of claim 72, said method comprising introducing into said subject said genetic sequence flanked by methylation-preventing sequences to thereby replace the silenced genetic sequences or introducing a protein or non-proteinaceous product which replaces the protein encoded by the silenced genetic sequence.

81. The method of claim 80 wherein the potentially-methylated sites are CpG or CpNpG nucleotides within the recognition sequence of the methylation-sensitive enzyme.

82. The method of claim 80 wherein the methylation-sensitive enzyme is selected from the list comprising AatII, AciI, AclI, AgeI, AscI, AvaI, BamHI, BsaAI, BsaH1, BsiE, BsiW, BsrF, BssHII, BstBI, BstUI, Cla1, EagI, HaeII, HgaI, HhaI, HinPI, HpaII, MloI, MspI, NaeI, NarI, NotI, NruI and PmlI.

83. The method of claim 82 wherein the methylation-sensitive enzyme is HpaII or its functional equivalent.

84. The method of claim 83 wherein the methylation-sensitive enzyme is HpaII.

85. The method of claim 80 wherein the amplification reaction is selected from polymerase chain reaction (PCR), ligase chain reaction (LCR), gap filling LCR (GLCR), Qβ replicase; stand displacement amplification (SDA); self-sustained sequence replication (3SR) nucleic acid sequence-based amplification (NASBA).

86. The method of claim 85 wherein the amplification reaction is PCR.

87. The method of claim 86 wherein the amplification reaction is real-time PCR.


SEQUENCE LISTING (38 sequences)

SEQ ID NO: 1 - DNA, 10 bp, Artificial
  Primer
tttgcccgga

SEQ ID NO: 2 - DNA, 10 bp, Artificial
  Primer
tggaccggtg

SEQ ID NO: 3 - DNA, 10 bp, Artificial
  Primer
acccggtcac

SEQ ID NO: 4 - DNA, 10 bp, Artificial
  Primer
aacccgggaa

SEQ ID NO: 5 - DNA, 10 bp, Artificial
  Primer
ttcccgggtt

SEQ ID NO: 6 - DNA, 10 bp, Artificial
  Primer
tttgcccggt

SEQ ID NO: 7 - DNA, 10 bp, Artificial
  Primer
cccggcataa

SEQ ID NO: 8 - DNA, 10 bp, Artificial
  Primer
cacccggatg

SEQ ID NO: 9 - DNA, 10 bp, Artificial
  Primer
tcagtccggg

SEQ ID NO: 10 - DNA, 10 bp, Artificial
  Primer
tgccggcttg

SEQ ID NO: 11 - DNA, 10 bp, Artificial
  Primer
ccccggtaac

SEQ ID NO: 12 - DNA, 10 bp, Artificial
  Primer
cagtgccggt

SEQ ID NO: 13 - DNA, 10 bp, Artificial
  Primer
accggcttgt

SEQ ID NO: 14 - DNA, 10 bp, Artificial
  Primer
gtccggagtg

SEQ ID NO: 15 - DNA, 10 bp, Artificial
  Primer
acaccggaac

SEQ ID NO: 16 - DNA, 10 bp, Artificial
  Primer
cccggatggt

SEQ ID NO: 17 - DNA, 10 bp, Artificial
  Primer
aagaccggga

SEQ ID NO: 18 - DNA, 10 bp, Artificial
  Primer
tcccggtgag

SEQ ID NO: 19 - DNA, 10 bp, Artificial
  Primer
gaatccggca

SEQ ID NO: 20 - DNA, 10 bp, Artificial
  Primer
acccggaaac

SEQ ID NO: 21 - DNA, 10 bp, Artificial
  Primer
tgccggttca

SEQ ID NO: 22 - DNA, 10 bp, Artificial
  Primer
agccgggtaa

SEQ ID NO: 23 - DNA, 10 bp, Artificial
  Primer
cccggataca

SEQ ID NO: 24 - DNA, 10 bp, Artificial
  Primer
cccggaagag

SEQ ID NO: 25 - DNA, 10 bp, Artificial
  Primer
ctaccggcac

SEQ ID NO: 26 - DNA, 10 bp, Artificial
  Primer
acctccggtc

SEQ ID NO: 27 - DNA, 10 bp, Artificial
  Primer
ctccggatca

SEQ ID NO: 28 - DNA, 10 bp, Artificial
  Primer
tttccgggag

SEQ ID NO: 29 - DNA, 10 bp, Artificial
  Primer
aggccggtca

SEQ ID NO: 30 - DNA, 10 bp, Artificial
  Primer
caaccggtct

SEQ ID NO: 31 - DNA, 10 bp, Artificial
  Primer
ccgccggtaa

SEQ ID NO: 32 - DNA, 10 bp, Artificial
  Primer
tccgggactc

SEQ ID NO: 33 - DNA, 396 bp, Artificial
  Markers
tttcccgggttgcctatcgcgtacttggcacacagttaagtgctcgagaaattgctaaacactcaacgaatatttaccagaatacatatttatgctccaccgttgctgaaagaatttcgactaacccttctttaagtctctcttaccctcctgtactattaacaccattgtaagtaggcagtggagctacatttgcagggagagagaagagcgaaaaattggcgcgcagagctgtaaagcgtttttctccaggtcacaccgcagcctgtaaatttgtgacacttggaccgattatgggacagcttgcaaacaaattatctgcaaactaacgaagcctatttttaaagatccctgttcctcgcagctctaggcgatgacatcagagaacccgggaaa

SEQ ID NO: 34 - DNA, 259 bp, Artificial
  Markers
taacccgggaaataaattgagaggttttttaggcttactgcatgttggctattctgctccatagagggaggttcctgtttccttgctttgcctcccctagtcacgccagataaacattttccaaaagcaatttgacgcgctaaatttaagtatctcccaagacacagggttcctaagtaacactgagcccttgcagcagaaacccagtagggtccatgggcattgcatgtttaaaggtgttcttgggattcccgggtta

SEQ ID NO: 35 - DNA, 299 bp, Artificial
  Markers
ttggaccggtgtccagtcaagggaataaggaggtgcattcgatgactggagttagggcactttaaatgcaggactgaaaatcagaaaggacgtgttctcccccgagaaagcagagagagcagagcaaagcgagaagaattggggatgcttgcctcatccaggttataaaaaaaaaaaaaaggcctccagccttgggtgatggcaatgttgtttgtgctgttacaatgcagcagattttacattttgccctctttgcaaacctattagcggacactgctgaagttcccacaccggtccaa

SEQ ID NO: 36 - DNA, 103 bp, Artificial
  Markers
tcacccggatgacatgtctgttgacaattttaccaggtaattaaggaggtaatttcctacacggtataggtgaattgtccagtctttaaacacatccgggtga

SEQ ID NO: 37 - DNA, 1750 bp, rpA2 promoter element
caggcgcccgccaccacacccagctaattttttatatttttagtagagatggggtttcaccgtgttagccagcatggtcttgatctcctgatctcgtgatcagggaaatctgtttaattaaaacacgctttgcgatttctgcccagtgctctgaatgtcaaagtcaagagtaggaggaagaagaaagaagaaagcagggtgcagtggctgacgcctgtaatcccaacactttgggaggccaaggtgggtggatcacttgaggtcaggagttcgagaccagcctgaccagcagggcgaaacccccgtctctactaaaaatacaaaaattagcagggcgtggttgcacgtacctgtaatcccagctacgccggaggctaaggcaggaaaatcgcttgaacccgggaggcggaggttgcagtgaggtgagatcgcaccattgcactccagcctgggcaacaagagcaaaactcagtttccgaaaaaaaaaaaaaaaggaaaaagaagagaagaaaaaaacacacagtcctggagcccaaaggccatgtctatcttacttaaccattgaatcccagtggccagcacagggccaggaacaaagaaggcataaacaattcttttttttttttttttttttttttgagtcgcagtctctctctgccacctaggctggagtgcagtggcagcatcacagctcactgcagcctggacaccccagggccaggtgatcctcccaccttagccaccccagtagctgggaccccaggcacttgccaccacaccagactaatttttaaaaatattttttgagagagggtctcactatgttgtccaggctggtctcaaacttccagcctcaagcggtcctcctgcctcagaccccatttgctgggtttacaggcatgagccacagcacctgctaatttttcttaaatacataaatgaacataaaattctaacaatgcatgagtattttgaggaaggaactgacaaaatgttccactccctatgggaggcacgttatatgaagaattatgaaaaatggtcgaaatgactggagaggccaagcctggatgagactgggatggggacaggtgcgggacgaggggcaccaccctcacatctttcacaagtctgtcataggcaagagggcgtaggtttctcacagccccactggggagaatcggcaccattggtggcattacacgaagagaatgtgacctcctatgtaaaagaacaagcaactccacgcggtgctgtgaggctagtgctgcgagtccctgaggtgcgcaattcccgcacgaccgtgggtgggaaacaccgaagccaaaactccgctacagccctttagatgaaggcgtcgtctgattggtgatagtttggcgcgaacctgagcacgccgaacaaaggaagtgacggcagaagtcgcgcacttgacgagggtgggatcacacggcgctgcgtcgcggtagtattgttctgattggttgatttcttgcgataccgctctgccagccccttgcttccgctagtgcggagggttttgcccttcgtaaagatggccgcggaggcttttggagccaactgggagcgcagtacgcgttttctggagcatgggcagaggagacaggaacaagcgtagcatccgtgagcaccgattggctgaagcgagcaccccgggagctgactggctccgccattcgcgggaaggcgtttgtggtgccagagaaaagtagccagagcggcgcagtgg

SEQ ID NO: 38 - DNA, 1510 bp, rbp21 promoter element
ctgcagagaggatgacccttgtcaccaccgtcatgtacgaggctgcttcaccactgcctcactgccaccagcgtctcccgccgcgtgcaatacaagaagaaacatcgaacggtcatataaggtaagacccactaccgatttaacctatcattcccacaatctaatccacttatttctcttcccatgatcttatcctctcatttctcctcactacttttgcatttgtaggaaacacaatgacaccgtcgaagaaagctggtggagcaccgtagccagcaatcaccaaaacacagaggggaggaggtcggcagcggccatgcggacggcgatgagacaacgcgacgcaaagagggaggaggacgttggcgatcatgctggtgttggcggaggaggtcactggccatgcgaatgacagcggggcagcgcaacacaaaaaggggggaggatgccggcgaccacgctagtaccatgaagcaagatgatgtgaaagggaggaccggacgagggttggacctctgccgccgacgtgaagagcgtgatgtgtagaaggagatgttagaccagatgccgacgcaacttagccctgcaagtcacccgactgcatatcgctgcttgccctcgtcctcatgtacacaatcagcttgcttatctctccatacttgtcgtttgtttcccgtggccgaaatagaagaagacagaggtgggttttgttggagagttttagtggtattgtaggcctatttgtaattttgttgtactttattgtattaatcaataaaggtgtttcattctattttgactcaatgttgaatccattgatctcttggtgttgcactcagtatgttagaatattcattccgttgaaacaatcttggttaagggttggaacatttttatctgttcggtgaaacatccgtaatattttcgttgaaacaatttttatccgacagcaccgtccaacaatttacaccaatttggacgtgtgatacatagcagtccccaagtgaaactgaccaccagttgaaaggtatacaaagtgaacttattcatctaaaagaccgcagagatgggccgtggccgtggctgcgaaacgacagcgttcaggcccatgagccatttattttttaaaaaaatatttcaacaaaaaagagaacggataaaatccatcgaaaaaaaaaaactttcctacgcatcctctcctatctccatccacggcgagcactcatccaaaccgtccatccacgcgcacagtacacacacatagttatcgtctctccccccgatgagtcaccacccgtgtcttcgagaaacgcctcgcccgacaccgtacgtgcgccaccgccgcgcctgccgcctggacacgtccggctcctctcccgccgcgctggccaccgtccaccggctcccgcacacgtctccctgtctccctccacccatgccgtggcaatcgagctcatctcctcgcctcctccggcttataaatggcggccaccaccttcacctgcttgca